(19) United States
(12) Patent Application Publication
(10) Pub. No.: US 2025/0336009 A1
(43) Pub. Date: Oct. 30, 2025

(54) METHODS AND SYSTEMS FOR GENERATING SPATIAL MAPS FOR AGRICULTURE SITES AND IMPLEMENTING AGRICULTURE INTERVENTIONS ACCORDING TO GENERATED ARCHETYPES

(71) Applicant: Biome Makers Inc., Davis, CA (US)

(72) Inventors: Sam Röttjers, Eygelshoven (NL); Ivan Martin, Salamanca (ES); Diego Rodríguez de Prado, Valladolid (ES); Beatriz Garcia-Jimenez, Madrid (ES); Alberto Acedo Becares, Valladolid (ES); Adrián Ferrero Fernández, La Bañeza (ES); Blas Manuel Benito de Pando, Castell de Ferro Granada (ES); Marko Budinich, Nantes (FR)

(73) Assignee: Biome Makers Inc., Davis, CA (US)

(21) Appl. No.: 19/188,516

(22) Filed: Apr. 24, 2025

Related U.S. Application Data
(60) Provisional application No. 63/639,952, filed on Apr. 29, 2024.

(51) Int. Cl. G06Q 50/02; G01N 33/24

(52) U.S. Cl. CPC G06Q 50/02; G01N 33/245

(57) ABSTRACT

Systems and methods for generating high-resolution spatial maps of microbiome and physicochemical indices for an agriculture site are provided. The spatial maps are generated from a limited/reduced number of physical samples acquired using a smart sampling tool provided by the systems and methods described. Insights for the agriculture site can be used to guide selection and application of interventions, according to various intervention archetypes, based upon the customized needs of the agriculture site. Performance of the agriculture site can thus be enhanced in an unprecedented, accessible, and sustainable manner.

### CROSS-REFERENCE TO RELATED APPLICATIONS

This application claims the benefit of U.S. Provisional Application No. 63/639,952 filed on 29 Apr. 2024, which is incorporated in its entirety herein by this reference.

### FIELD OF THE INVENTION

The disclosure generally relates to systems and methods for generating spatial maps for agriculture sites, and designing and/or applying agricultural interventions according to generated archetypes.

### BACKGROUND

Currently, agricultural producers often rely on expert knowledge from agronomists or on manufacturer recommendations for decisions regarding usage of agriculture interventions to produce a desired outcome. While scientific trials may be used in marketing materials to support product claims, those tend to be limited in number and generally showcase product use in ideal situations, from a highly-biased perspective. Often, such scientific trials are not peer reviewed, and time scales for verifying results attributed to a particular intervention are long in the field of agricultural production. Thus, there is a significant amount of waste in time and resources when applying a particular agriculture intervention to a site.

Additionally, technologies in fields relating to precision agriculture are limited in their abilities, as attributed to high capital requirements, high sampling costs, variable soil conditions, and data scarcity, which ultimately limit predictive capabilities and scalability of applying solutions to improve agriculture site performance in a sustainable manner.

As such, there is a need for an independent, accurate, and massively-data-driven approach to generating, guiding use of, and implementing agriculture interventions for end users, while providing highly effective tools for understanding agriculture sites.

### BRIEF DESCRIPTION OF THE FIGURES

FIG. 1A depicts an embodiment of a workflow of a method for generating, evaluating, and applying agricultural intervention archetypes.

FIG. 1B depicts an embodiment of a workflow of a method for generating, evaluating, and applying agricultural intervention archetypes.

FIG. 1C depicts an embodiment of a workflow of a method for generating spatial maps of microbiome and physicochemical features at one or more agriculture sites.

FIG. 2 depicts a schema showing phases of system architecture for generating, evaluating, and applying agricultural intervention archetypes.

FIG. 3 depicts a first example application of a system and method for generating, evaluating, and applying agricultural intervention archetypes.

FIG. 4 depicts a second example application of a system and method for generating, evaluating, and applying agricultural intervention archetypes.

FIG. 5 depicts a schematic of an embodiment of a system for generating, evaluating, and applying agricultural intervention archetypes.

FIG. 6 depicts a schematic of integrated components of a mapping system for generating and providing agriculture site insights.

FIG. 7 depicts a schematic of a zonification algorithm used to guide optimization of agriculture site sampling, where the left panel represents the agriculture site subregions, the center panel summarizes the machine-learning-based clustering algorithm used to return candidate sampling zones, and the right panel shows the resulting recommended sampling zones and locations.

FIG. 8 depicts exemplary database components of a mapping predictors catalog used to provide quality control for systems structured for generating and providing agriculture site insights, where the left panel depicts an organization of the data schema, and the right panel lists the remote-sensing and topographic variables stored in the catalog.

FIG. 9 depicts an exemplary returned mapping output, provided at a user interface, where the returned mapping output depicts a microbiome map representing the metabolism of nitrogen.

FIG. 10 depicts a workflow of steps performed by subsystems used to generate a microbiome and/or physicochemical map for a given agriculture site.

FIG. 11 depicts a workflow of steps performed by an embodiment of a smart sampling subsystem used to facilitate generation of spatial maps of agriculture sites.

FIG. 12 depicts a workflow of steps performed by an embodiment of a mapping subsystem used to generate spatial maps of agriculture sites.

FIG. 13B depicts an exemplary flow of a method associated with system 800.

FIG. 13C depicts an application of the invention(s) associated with the system 800.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference.

Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### DETAILED DESCRIPTION OF THE INVENTION(S)

The following description of the preferred embodiments of the invention is not intended to limit the invention to these preferred embodiments, but rather to enable any person skilled in the art to make and use this invention.

### 1. Benefits

The invention(s) described can confer several benefits over conventional systems, methods, and compositions.

In particular, the systems and methods described provide significant advancements in microbiome and physicochemical mapping, at farm-scale, at resolution scales smaller than a farm unit, and/or at resolution scales larger than a farm unit. By integrating technology for characterizing agriculture site microbiome and physicochemical aspects with: a) high-resolution satellite imagery and topographic data, b) high-performance raster processing, and c) spatial modeling, the invention(s) described provide high-accuracy soil microbiome maps with reduced sampling requirements. As such, the inventions provide end-users with agriculture site insights, with extremely reduced sampling requirements (e.g., in relation to numbers of samples required, in relation to sampling time, in relation to sampling resources required, etc.) The inventions described thus address key challenges in precision agriculture, where current techniques are subject to high sampling costs, variable soil conditions, and data scarcity. The integrated systems and methods described further enhance predictive capabilities (e.g., in relation to microbiome and physicochemical feature trends for evaluated agriculture sites), reduce errors associated with agricultures site characterization, and improved scalability of responses to returned insights, thereby providing a new and useful tool for farmers, agronomists, and researchers seeking data-driven insights into soil health and productivity.

The inventions, including systems and methods for microbiome and physicochemical mapping, further address major challenges in precision agriculture with a strong focus on efficiency, automation, and scalability. The integration of high-resolution remote sensing, spatial modeling, and proprietary microbiome data is novel, and outperforms existing approaches. Particularly, the inventions introduce multiple innovations that significantly improve soil mapping accuracy and efficiency:

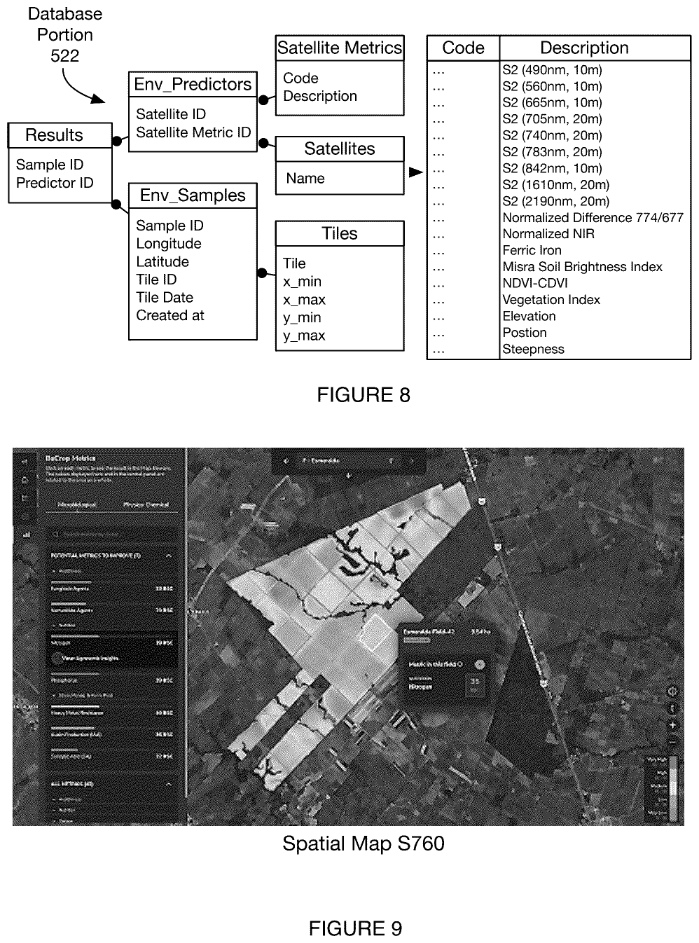

Reduced Sampling Requirements: as described below, by inclusion of a mapping predictors catalog (e.g., as depicted in FIG. 8), the inventions described reduce the required sampling requirements, thereby making mapping of microbiome and physicochemical features at an agriculture site more accessible and cost-effective. In examples, the inventions can reduce a number of samples required to achieve maps having the resolution attributes described, by: 20%, 30%, 40%, 50%, 60%, 70%, 80%, 90%, or greater, in comparison with current techniques for generating agriculture site maps from acquired agriculture site samples. As such, the sampling subsystem reduces a number of samples required to generate the spatial map by percentages described, in comparison with a process that omits involvement of the sampling subsystem.

For an exemplary site having a dimension of more than 0.5 acres, more than 1 acre, more than 2 acres, more than 3 acres, more than 4 acres, etc., the number of sampling locations can be reduced by the percentages described above.

Enhanced Accuracy: The addition of relevant samples from the mapping predictors catalog (described in relation to FIG. 8 below) to the agriculture site samples increases the training sample pool during training of models described, and thereby increases model accuracy by at least 10%, by at least 15%, by at least 20%, by at least 30%, by at least 40%, or greater depending on the total number of samples available from the agriculture site. Accuracy performance improvements are characterized in relation to performance of systems and methods that implement only agriculture site sampling, or only mapping predictor catalog data. Notably, accuracy gains are inversely correlated with number of samples from the agriculture site. Model accuracy can be determined by comparing predicted attributes (e.g., microbiome attributes, physicochemical attributes) across mapping locations of a generated map, with actual attributes (e.g., microbiome attributes, physicochemical attributes) across mapping locations of a generated map. In examples, relevant accuracy metrics can include a Root Mean Squared Error Percentage (RMSE %), which quantifies the average differences between observed and predicted values; a Probability of RMSE % Being Better Than Chance (i.e., RMSE % prob), which quantifies the probability of obtaining RMSE % smaller than the observed by chance, and therefore relates to the credibility of the RMSE % score; and/or other suitable accuracy metrics.

Cloud Removal Algorithm: The addition of an automated cloud removal process for returned data from the systems and methods described increases the availability of high-quality imagery associated with a generated map, thereby enhancing spatial data consistency across different returned maps and increasing data availability in regions with high cloud cover. The cloud removal algorithm aggregates portions of several images to generate combined high-quality image for an area of interest.

High-Resolution Topography Integration: The inventions implement high-resolution topographic data as a predictor for generation of maps with enhanced mapping realism (e.g., in hillside agricultural regions, in areas with variable terrain, etc.).

Optimized Database Usage: The inventions implement architecture that streamlines data requests to databases of the platform, thereby minimizing operational costs while maintaining high processing efficiency.

Automated Sample Selection and Processing: Embodiments, variations, and examples of the mapping predictors catalog described dynamically identify and process relevant samples (e.g., soil samples, other samples that can provide microbiome and/or physicochemical information) from agriculture sites, enabling more scalable and adaptable mapping workflows.

Additionally, the inventions address several problematic aspects of current approaches to generating, guiding, and/or implementing agriculture interventions, with respect to end users (e.g., agriculture producers, agriculture site managers, any entity in the supply chain, etc.). Agriculture intervention application at an agriculture site can be guided or informed by outputs of precision mapping tools described above.

The disclosure thus also provides an artificial intelligence (AI)-based decision support system to guide in the application, design or research of any agricultural intervention, such as a biostimulant or management practice strategy. The invention(s) use a combination of computer-readable domain knowledge and test data generated from site sampling in coordination with applied interventions, to estimate the effects of the intervention on soil microbiome characteristics of interest. Generated and refined AI models can then be applied to extrapolate estimated effects: i) to locations where a microbiome sample has been collected, even if no data are available for a specific intervention; and to ii) compare the effects of distinct interventions with respect to specific measures of efficacy, in an unprecedented manner.

The invention(s) leverage large databases (e.g., proprietary databases) of agriculture site data pertaining to crops, conditions, and interventions, to estimate product efficacy in a manner that is significantly less biased or unbiased, in comparison to data generated by product providers. System architecture is structured such that misrepresentation of product efficacy is less likely and/or eliminated because the system rewards predictable poor performance. If a product is consistently predicted to perform poorly in a specific setting, the system uses this information to support decision making with respect to implementation of suggested agriculture intervention(s). Recommended intervention(s) and/or supporting rationales can then be provided to the end users, which represents a fundamental improvement over current solution approaches, where a client may not have access to this level of information. Implementation of artificial intelligence model architecture according to the invention results in performance at a level that cannot be achieved in the human mind, where the model architecture is structured to transform large amounts of information into actionable insights that do not require expert knowledge.

The invention(s) also provide benefits in that generated predictions and implemented agriculture interventions can deviate from expectations based on conventional knowledge. In one exemplary use case, the invention(s) can return recommended biostimulant products that improve nutritional statuses of the soil, in comparison with pesticide use, if usage of the biostimulant is predicted to have a similar impact on soil health but improved effects on the soil ecology. This data-driven approach allows users of the invention(s) to explore interventions in novel ways, beyond marketing claims presented by manufacturers.

The invention(s) also return outputs that are personalized and customized to each site, given that the invention(s) use characteristics of the location(s)/agriculture sites of interest to the end user or other relevant entity. For instance, If weather conditions have a major impact on intervention efficacy, locations with optimal weather conditions for that intervention will be more likely to have positive outputs for the specific intervention. Similarly, many other characteristics are included as inputs to models associated with the invention(s), including microbiome-derived markers of soil health or nutrient metabolism. This is especially relevant to novel biologics in agriculture, which often promise sustainability but cannot always deliver consistent performance. The system inventions are thus able to suggest locations where such biologics may perform well and can therefore potentially improve their consistency.

The invention(s) are also able to flexibly provide recommendations according to a set of one or more multiple indicators of efficacy. For some interventions, efficacy may be reported as changes in nutrient status, while others may be marketed to improve disease status. As such, models of the inventions can be tuned and returned to provide specific interventions for specific desired outcomes, in relation to efficacy. There is no limitation on the number of indicators that can be returned, meaning that the returned outputs of the invention(s) can be flexibly tailored to user interests. This also represents a novel improvement over the current situation, where clients are often limited to the information provided by the manufacturer or the experience of experts they consult. In other relevant examples, the invention(s) can generate outputs pertaining to the impact of pesticides on markers of soil ecological health. Such information is both difficult to find and challenging to interpret, but may be highly relevant to the user.

The invention(s) provide systems and methods for prediction of various agriculture site and crop features, which are useful in downstream applications in relation to recommending or implementing various agriculture inputs and/or management practices to improve productivity or maintain health of the agriculture site.

Additionally, in embodiments, the invention(s) provide methods for determining microbiome-associated or-derived properties and/or properties derived from network properties in local microbial, fungal, and/or other organism communities, and to use them to assess the impact of different agricultural inputs and/or practices (e.g., farming practices).

The invention(s) can further provide methods and systems for evaluating, guiding, and/or executing implementation of various agricultural inputs and/or management practices for enhancement of yield and/or a yield effect as a selected effect (e.g., in relation to specific soil types and/or for specific crops), enhancement of nutritional status. improvement of agriculture site characteristics (e.g., with respect to health, with respect to sustainability), improvement of sustainability (e.g., with respect to net carbon metrics, with respect to carbon capture metrics, with respect to other resource use and waste aspects, etc.).

Additionally, the inventions described provide systems and a platform including architecture for agriculture sample extraction and processing, which provide improved tools for monitoring, forecasting, and responding to events (e.g., changes in productivity, events associated with management practices, environmental perturbations, product-induced perturbations, etc.) associated with one or more agricultural sites. Additionally or alternatively, the inventions can assess implementation of a plant variety and/or a seed variety at an agriculture site.

Additionally, the inventions apply outputs of the analyses to effect one or more actions (e.g., agriculture interventions) to maintain or improve the natural ecological site conditions according to various metrics of efficacy, where the metrics can be weighted differently for each user, thereby providing practical applications of the method(s) and models involved.

Additionally, the inventions involve collection of samples from various agricultural sites, processing of samples to extract data features, application of one or more transformations to the data features to generate modified digital objects, create improved training data sets for machine learning/classification algorithms, and iteratively train the machine learning/classification algorithms, such that agriculture site statuses can be returned upon processing subsequent samples hitherto unseen by the algorithm.

In applications, the inventions can contribute to significantly increased yields of major/important crops (e.g., rice, wheat, soybeans, maize, potatoes, etc.) to improve global food production in relation to anticipated world population increases. Taking into account the effects of human intervention on soil ecology, the inventions can provide recommendations (management, treatment, etc.) that increase yield preserving ecology. In particular, using potato crops as an example, applications of the inventions can characterize yield (e.g., maximum potential yield) of potato crops based on current inputs and management practices, and/or recommend or implement agricultural inputs and improved practices for enhancement of yield and/or agriculture site characteristics.

Additionally or alternatively, the invention(s) can confer any other suitable benefit in any crop.

### 1.1 Exemplary Definitions

Terms provided herein are given as exemplary definitions. Additional terms are provided throughout the written description.

Agricultural intervention: any defined intervention applied in agriculture. Agricultural interventions or agriculture interventions are commonly used to describe application of agrochemicals, but may also describe management strategies including, but not limited to: organic management practices (e.g., integrating cultural, biological, and mechanical practices that foster cycling of resources, promote ecological balance, and conserve biodiversity without use of synthetic fertilizers, sewage, irradiation, and genetic engineering); non-organic management practices; use of synthetic fertilizers; use of natural fertilizers; biodynamic management practices (e.g., with generation of their own fertility through composting, integrating animals, cover cropping, and crop rotation); and conventional management practices (e.g., with standard farming systems, using a variety of synthetic chemical fertilizers, pesticides, herbicides and other continual inputs, etc.).

Biologics: agricultural biologics or biologicals are products containing or derived from living organisms, e.g. fungal spores or metabolites produced by living organisms Decision support system: system and software intended to improve decision-making capabilities by collecting and presenting information in useful ways.

Intervention effect: prediction for changes caused by a specific intervention in a specific location, expressed as a score, level or rank which describes the confidence with which the system predicts the change for a specific trait. The trait can be equivalent to an agronomic index or derivative of one or multiple agronomic indices, embodiments, variations, and examples of which are described in U.S. application Ser. No. 17/665,332 titled “Methods and Systems for Generating and Applying Agronomic Indices from Microbiome-derived Parameters” and filed on 4 Feb. 2022.

Intervention efficacy: performance measure of interventions across multiple locations, expressed as the ability of interventions to produce desirable changes.

Intervention archetypes: a description of an intervention which can be used to describe many other interventions; for instance, a broad-spectrum pesticide can be used as an archetypal intervention with traits typical of a large set of agrochemicals.

Location: a geographical unit where one or more samples have been collected.

Location characteristics: data used to describe properties of a location;

characteristics can include environmental data, but can also be derived from microbiome samples or other sources (e.g. traits related to plant life).

Additionally, the terms microbiome, microbiome information, microbiome data, microbiome population, microbiome panel and similar terms are used in the broadest possible sense, unless expressly stated otherwise, and would include: a census of currently present microorganisms, both living and non-living, which may have been present months, years, millennia or longer; a census of components of the microbiome other than bacteria and archaea (e.g., viruses, microbial eukaryotes, etc.); population studies and characterizations of microorganisms, genetic material, and biologic material; a census of any detectable biological material; and information that is derived or ascertained from genetic material, biomolecular makeup, fragments of genetic material, DNA, RNA, protein, carbohydrate, metabolite profile, fragment of biological materials and combinations and variations of these.

“Nucleic acid,” “oligonucleotide,” and “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. The term nucleic acid is used interchangeably with gene, cDNA, and mRNA encoded by a gene.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media.

Information may be transferred between a system of interest and a medium, between computers, or between computers and the computer-readable medium for storage or access of stored information. Such transmission can be electrical, or by other available methods, such as IR links, wireless connections, etc.

### 2. Methods

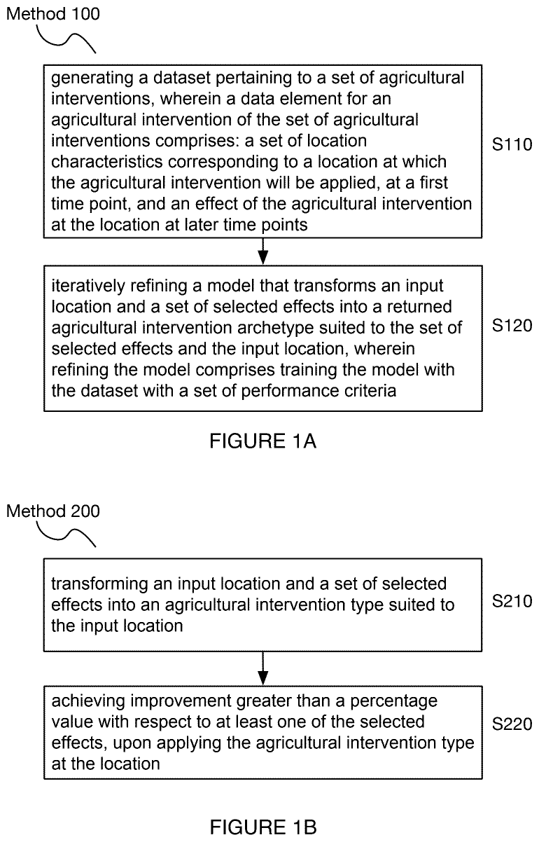

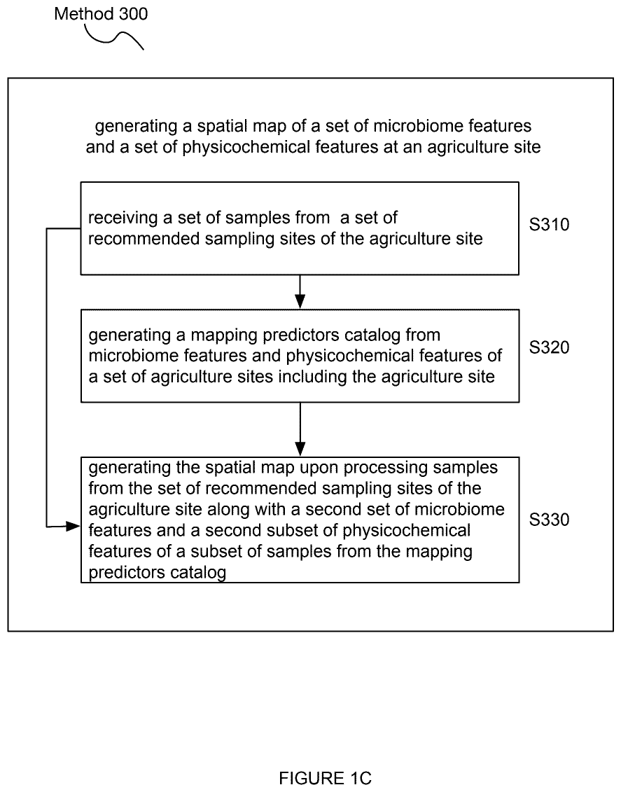

As shown in FIG. 1A, an embodiment of a method 100 includes: generating a dataset pertaining to a set of agricultural interventions, wherein a data element for an agricultural intervention of the set of agricultural interventions comprises: a set of location characteristics corresponding to a location at which the agricultural intervention will be applied, at a first time point, and an effect of the agricultural intervention at the location at a second time point S110; and iteratively refining a model that transforms an input location and a set of selected effects into a returned agricultural intervention archetype suited to the set of selected effects and the input location, wherein refining the model comprises training the model with the dataset with a set of performance criteria S120.

As shown in FIG. 1B, an embodiment of a method 200 includes: transforming an input location (upon receiving an agriculture site input location) and a set of selected effects into an agricultural intervention type suited to the input location S210; and achieving improvement greater than a percentage value with respect to at least one of the selected effects, upon applying the agricultural intervention type at the location S220.

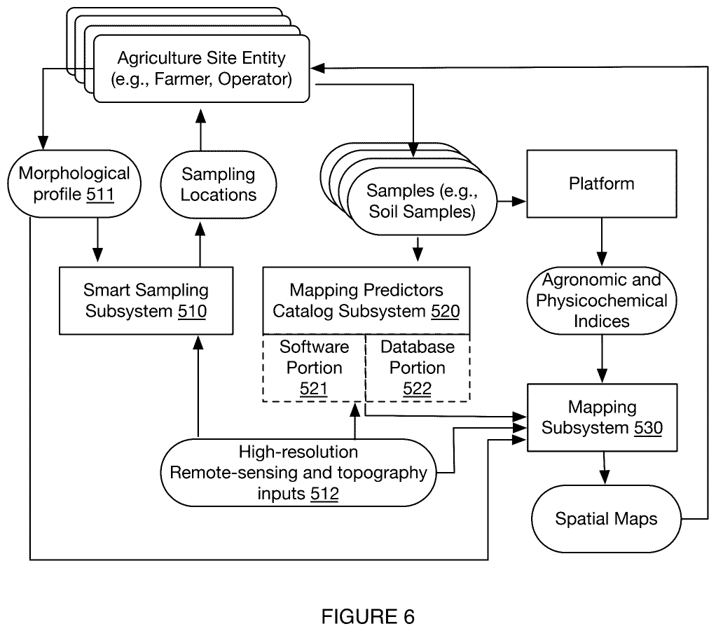

As shown in FIG. 1C, an embodiment of a method 300 includes: generating a spatial map of a set of microbiome features and a set of physicochemical features at an agriculture site, wherein generating the spatial map comprises: receiving a set of samples from a set of recommended sampling sites of the agriculture site, the set of samples determined from a sampling subsystem structured to generate an analysis of heterogeneity of the agriculture site, and to return the set of recommended sampling sites for the agriculture site upon processing the analysis with remote-sensing data and topographic data and contextual information for the agriculture site S310; generating a mapping predictors catalog from microbiome features and physicochemical features of a set of agriculture sites including the agriculture site S320; and generating the spatial map upon processing samples from the set of recommended sampling sites of the agriculture site along with a second set of microbiome features and a second subset of physicochemical features of a subset of samples from the mapping predictors catalog S330. Aspects of system components associated with the method 300 are shown in FIG. 6.

The methods 100, 200, 300 function to achieve groundbreaking performance with respect to desired effects at various agriculture sites, using a data-driven and microbiome-focused approach that is based on information that is normally difficult to access or use. The methods 100, 200 300 also function to provide output agricultural interventions that are customized to a set of desired input features, including, but not limited to: location, crop type, sample type, and desired effects. The methods 100, 200 involve automatic curation of input data driven by expert domain knowledge, providing standardized and quality-controlled model behavior, which can then be applied globally by extrapolation of model results due to the refinement processes described herein. The methods 100, 200 thus provide a high flexibility solution to achieving specific goals for an agriculture site because insights can be customized to the needs of the user by focusing on intervention effects of interest to the user, in use cases relevant to the user.

The methods 100, 200, 300 can be implemented by embodiments, variations, and examples of system elements described in one or more of: U.S. application Ser. No. 17/119,972 filed on 11 Dec. 2020; U.S. application Ser. No. 17/587,016 filed on 28 Jan. 2022; U.S. Application No. 176/665,332 filed on 4 Feb. 2022; and U.S. application Ser. No. 17/703,095 filed on 24 Mar. 2022, each of which is incorporated herein in its entirety by this reference.

### 2.1 Method-Data Generation and Curation

Step S110 recites: generating a dataset pertaining to a set of agricultural interventions, wherein a data element for an agricultural intervention of the set of agricultural interventions comprises: a set of location characteristics corresponding to a location at which the agricultural intervention will be applied, at a first time point, and an effect of the agricultural intervention at the location at a second time point.

Step S110 can involve curation of domain knowledge to define initial archetypes for various agricultural interventions, which are then pre-processed to define a set of input archetypes for model training, based upon traits of interest. In one example of Step S110, curation of domain knowledge can involve an algorithmic approach to define initial archetypes for various agricultural interventions, followed by manual curation (e.g., by an expert) to pre-process the initial archetypes based on traits of interest. These pre-processed archetypes are then used to programmatically curate the input data used by the decision support system, with respect to model training and refinement.

Location characteristics can include geographical coordinates, environmental information, sample type, sample source, location history (e.g., with respect to management practices), soil compositional features, and/or other location characteristics.

Step S110 can additionally or alternatively include extraction and processing of samples from one or more agriculture sites, in coordination with applied agricultural interventions corresponding to various archetypes. In variations, the set of samples can include multiple samples or a single sample for each location, with one or more time points of sample collection. In variations, the number of samples can be one sample, two samples, three samples, four samples, five samples, 10 samples, 15 samples, 20 samples, any intermediate number of samples, or greater than 20 samples.

In variations, the set of time points across which samples are acquired and analyzed can include two time points, three time points, four time points, five time points, 10 time points, 15 time points, 20 time points, any intermediate number of time points, or greater than 20 time points. In examples, the method 100 can return results with as few as two time points. The set of time points can be distributed across time scales on the order of seconds, minutes, hours, days, months, or years; however, the set of time points can additionally or alternatively be distributed across other suitable time scales.

Similarly, if multiple locations are involved, different locations can have any suitable distance relative to each other. Furthermore, the set of samples can include samples from any soil type and/or in association with any crop. Time points can be prior to treatment, during treatment, and/or after treatment with one or more agricultural interventions. Time points for biological treatments can be different than those for chemical treatments or other inputs/practices. For instance, time points for biological treatments can be spaced apart by one day, two day, three days, four days, five days, six days, one week, two weeks, three weeks, any intermediate value, or more than three weeks. Time points for chemical treatments can be spaced apart by one week, two weeks, three weeks, four weeks, one month, two months, three months, any intermediate value, or more than three months. Furthermore, variations of Step S110 can implement one time point as a benchmark, in order to assess various agricultural interventions where multiple time points are not needed.

Samples can be received from various portions of the agriculture site(s) and/or states of processing of crops or other products derived from the agriculture site(s). In embodiments, samples can be extracted from soil, another substrate, water used in agriculture (e.g., water run-off), from various portions of crops, from organisms interacting with crops (e.g., parasites, other symbiotic organisms, etc.), from consumable products (e.g., food, beverages, supplements, etc.) derived from crops, from other surfaces (e.g., conduits used to deliver water or nutrients to crops, etc.), and/or from other suitable sampling sites. The samples can include solid samples (e.g., soil, sediment, rock, food samples). The samples can additionally or alternatively include liquid samples (e.g., surface water, sub-surface water, other liquids derived from crops, consumable products derived from crops, crop-derived products at various stages of processing, fermentation, curing, aging, drying, etc.). The samples can additionally or alternatively include gas samples (e.g., samples from gases obtained from a greenhouse, gases produced during processing of crops or crop-derived products, etc.). Samples can be taken from crop portions (e.g., reproductive portions, petals, leaves, fruits, roots, trunks, flowers, pollen, etc.) and/or from crops in various states of health (e.g., healthy states, distressed states, diseased states, etc.).

In variations, samples can include phyllosphere components (e.gl, leaves, stems, flowers); endosphere components (e.g., intracellular components of leaves, stems, roots, etc.); rhizosphere components (e.g., root, nodules, and surrounding soil components, etc.), and other components.

Sample volumes can range from 0.01 grams to 1 kilogram (or greater than 1 kilogram, less than 0.01 gram). Additionally or alternatively, sample volumes can range from 1 microliter to 1 liter (or greater than 1 liter, less than 1 microliter). Samples from different portions of a location, different portions of a crop, different portions or stages of a product being produced, and/or different sources can be combined and processed, or processed separately in Step S110.

In relation to Step S110, sample reception/collection can be performed using equipment (e.g., machinery, robotic apparatus configured to traverse a location in coordination with retrieval of the set of samples, other apparatus) and/or manually. In variations, sample reception/collection performed in Step S110 can use any one or more of: an instrument (e.g., scoop for soil, sharp instrument for extracting a portion of a crop specimen, etc.), a permeable substrate (e.g., a swab, a sponge, etc.), a non-permeable substrate (e.g., tape, etc.), a container (e.g., vial, tube, bag, etc.) configured to receive a sample from the agriculture site or associated crops, and any other suitable sample-reception element. In a specific example, samples can be collected from one or more of: soil, other crop-associated solids, water, other crop-associated liquids, gases, and a crop component (e.g., root, stem, leaf, flower, seed, other plant component, etc.). In relation to soil samples, samples can be extracted in relation to a reference point (e.g., distance from surface, distance from plant, etc.). In relation to plant components, samples can be taken from a reference (e.g., distance from leaf, distance, from node, distance along root, etc.). In variations in which multiple samples are taken, samples can be pooled (e.g., combined) or kept distinct.

In variations, the treatment states (e.g., first treatment state, second treatment state, third treatment state, control state, etc.) with respect to various agricultural interventions can be associated with a management practice, such as one or more of: organic management practices (e.g., integrating cultural, biological, and mechanical practices that foster cycling of resources, promote ecological balance, and conserve biodiversity without use of synthetic fertilizers, sewage, irradiation, and genetic engineering); non-organic management practices; use of synthetic fertilizers; use of natural fertilizers; biodynamic management practices (e.g., with generation of their own fertility through composting, integrating animals, cover cropping, and crop rotation); conventional management practices (e.g., with standard farming systems, using a variety of synthetic chemical fertilizers, pesticides, herbicides and other continual inputs, etc.).

In variations, the treatment states (e.g., first treatment state, second treatment state, third treatment state, control state, etc.) can be associated with inputs associated with the agriculture site(s) and/or crops from which samples are derived, including one or more of: a biological input including one or more of: a biostimulant, a biofertilizer, a biocontrol agent, a biopesticide, compost, and a biodynamic preparation (wherein the biological input is applied by one or more of: a broadcast spray, an in-furrow spray, seed treatment, application to soil with incorporation, and application to soil without incorporation, etc.); and another suitable input.

### 2.2 Method-Input Data Aspects

In relation to Steps S110 and S120 (described in more detail below), data pertaining to agricultural interventions is organized into archetypes containing general categories of agricultural interventions.

In variations, a first archetype can include broad-spectrum pesticides, where examples of broad-spectrum pesticides can include one or more of: organophosphates, carbamates, pyrethroids, neonicotinoids, dichlorodiphenyltrichloroethanes (DDTs), acetamiprids, beta-cyfluthrins, bifenthrins, dimethoates, fenpropathrins, formetanate hydrochlorides, malathions, methomyls, micronized sulfurs, naleds, neem oils, other oils, phosmets, pyrethrins, spirotetramats, zeta-cypermethrins, and other broad-spectrum pesticides.

In variations, a second archetype can include narrow-spectrum pesticides, where examples of narrow-spectrum pesticides can include one or more of: chitin inhibitors, abamectins, acequinocyls, afidopyropens, azadirachtins, bifenazates, buprofezins, chlorantraniliproles, copper bands, copper sulfates, cyflutmetofens, cyantraniliproles, fenbutatin oxides, fenpyroximates, flonicamids, flupyradifurones, hexythiazoxes, hydrated lymes, imidacloprids, iron phosphates, mating disruptors, metaflumizones, metaldehydes, methoxyfenozides, pyridabens, pyriproxyfens, sabadillas, smethoprenes, spinetorams, spinosads, spicrodiclofens, sodium ferric edtas, sticky materials, thiamethoxams, and other narrow-spectrum pesticides.

In variations, a third archetype can include intermediate-spectrum pesticides, where examples of narrow-spectrum pesticides can include one or more of: cryolites, diflubenzurons, and other intermediate-spectrum pesticides.

In variations, a fourth archetype can include biostimulants.

In variations, a fifth archetype can include fertilizers.

In variations, a sixth archetype can include complex fertilizers (e.g., micronutrients, nitrogen-phosphorous-potassiums (NPKs)).

In variations, a seventh archetype can include organic fertilizers.

In variations, an eighth archetype can include crop protectors.

In variations, a ninth archetype can include synthetic crop protection products.

In variations, a tenth archetype can include microbial and organic products with crop protection or biostimulant functionality.

In variations, an eleventh archetype can include products containing algae or algae-derived components.

In variations, a twelfth archetype can include products containing Bacillus.

In variations, a thirteenth archetype can include products containing plant endosymbionts.

In variations, a fourteenth archetype can include products containing Pseudomonas.

In variations, a fifteenth archetype can include products containing Trichoderma.

In variations, a sixteenth archetype can include organic management practices (e.g., integrating cultural, biological, and mechanical practices that foster cycling of resources, promote ecological balance, and conserve biodiversity without use of synthetic fertilizers, sewage, irradiation, and genetic engineering).

In variations, a seventeenth archetype can include non-organic management practices.

In variations, an eighteenth archetype can include biodynamic management practices (e.g., with generation of their own fertility through composting, integrating animals, cover cropping, and crop rotation).

In variations, a nineteenth archetype can include conventional management practices (e.g., with standard farming systems, using a variety of synthetic chemical fertilizers, pesticides, herbicides and other continual inputs, etc.).

In variations, a twentieth archetype can include products containing humic and fulvic acids.

In variations, a twenty first archetype can include biostimulants without microbial active ingredients.

Archetype-like configurations can also be generated using user-provided data. For example, a user could be a product manufacturer who wishes to compare predictions for their product(s) to relevant archetypes.

In relation to Steps S110 and S120 (described in more detail below), data pertaining to location characteristics associated with sampling and an applied agricultural intervention can include one or more of: environmental data, microbiome-derived data (e.g., taxonomic information, functional annotations, network properties, etc.), soil physicochemical characteristics, and plant-specific information. Embodiments, variations, and examples of location characteristics are further described in U.S. application Ser. No. 17/703,095 titled METHODS AND SYSTEMS FOR ASSESSING AGRICULTURE PRACTICES AND INPUTS WITH TIME AND LOCATION FACTORS and filed on 24 Mar. 2022, which is herein incorporated in its entirety by this reference. Alternatively, effects of agricultural interventions can be determined using other suitable methods and involve other values of other parameters.

In relation to Steps S110 and S120 (described in more detail below), data pertaining to effects of an applied agricultural intervention can include values of agronomic indices, embodiments, variations, and examples of which are described in U.S. application Ser. No. 17/665,332 titled METHODS AND SYSTEMS FOR GENERATING AND APPLYING AGRONOMIC INDICES FROM MICROBIOME-DERIVED PARAMETERS and filed on 4 Feb. 2022, which is herein incorporated in its entirety by this reference.

In relation to Step S110, data corresponding to initial intervention archetypes can be separated into units with multiple replicates and controls for each location. Data corresponding to initial intervention archetypes can, however, be organized in another suitable manner.

### 2.3 Method-Model Architecture

Step S120 recites: iteratively refining a model that transforms an input location and a set of selected effects into a returned agricultural intervention archetype suited to the set of selected effects and the input location, wherein refining the model comprises training the model with the dataset (e.g., all or a portion of the dataset of Step S110) with a set of performance criteria.

While an input location is described, inputs to the model can additionally or alternatively include any measure that can be used to describe a sample, and that allows determination of a distance (e.g., a quantitative distance) with respect to data elements. Examples of inputs (e.g. distance) can thus include taxonomic information (e.g. distance: beta diversity), environmental features (e.g. distance: Euclidean distance of environmental characteristics such as soil water content), qualitative metadata such as a crop grown in a location.

Model refinement is performed with all or a portion of the dataset described in Step S110, where data corresponding to initial intervention archetypes can be separated into units with multiple replicates and controls for each location. Data corresponding to initial intervention archetypes can, however, be organized in another suitable manner.

With respect to generation of the model, distances (e.g., Euclidean distance, Manhattan distance, Minkowski distance, etc.) between input data (e.g., input data comprising location data, used to test and refine the model) to archetype data is calculated, using mathematical transformations where appropriate. For each location represented in the input data, the nearest archetype location(s) and associated intervention effects (e.g., with respect to agronomic indices) are identified.

In relation to Step S120, selected effects according to which the model returns candidate agricultural intervention(s) can be user selected as user-defined selects, or automatically recommended as suggestions based upon input data aspects (e.g., recommended selected effects based upon input location).

In variations, models can incorporate an additional layer that provides the ability to generate recommendations (e.g., with respect to archetypes described) without requiring acquisition and processing of a microbiome sample. In such variations, the input to the model can be a delimited area (e.g., identified by a set of coordinates that define a multipolygon). The input is then used to search a set of environmentally similar microbiome samples in a database, in order to return the recommendations for those samples.

The model can be structured to return agricultural intervention scores, levels, or ranks that describe the confidence with which the model predicts an outcome for a returned agricultural intervention, based upon the selected effects. Selected effects can correspond to modulation of values of various agronomic indices or derivatives of multiple agronomic indices, embodiments, variations, and examples of which are described in U.S. application Ser. No. 17/665,332 titled METHODS AND SYSTEMS FOR GENERATING AND APPLYING AGRONOMIC INDICES FROM MICROBIOME-DERIVED PARAMETERS and filed on 4 Feb. 2022, which is herein incorporated in its entirety by this reference. Representative agronomic indices corresponding to selected effects can include one or more of:

2.3.1 Selected Effects in Relation to Agronomic Indices related to Biosustainability

1. Biosustainability: In examples, the invention(s) can generate and implement multiple (e.g., 3, less than 3, more than 3) metrics characterizing diversity of sample species and/or metabolic functions present in the sample(s) from the agricultural sites, as well as vulnerability of the system based on estimation of microbiome resistance (e.g., for instance, at an agriculture site input location). Biosustainability indices are biomarkers of the ecosystem in which a site is based, and related to management practices. In examples, three biosustainability indices can be generated:

1A. Biodiversity (species richness, evenness, and equilibrium of species): outputs can be generated from Shannon diversity characterization, based on taxonomic assignment. However, other outputs can additionally or alternatively be generated based upon evaluation of richness, phylogenetic entropy (e.g., based on a proprietary database of soil samples), or any other method(s).

1B. Functionality (capability of communities to perform one or more functions): outputs can be generated from Shannon diversity of the metagenomic functions predicted, but it could be any other diversity metric based on the functions.

1C. Resistance (stress adaptation, ability of communities or populations to remain unchanged when stressed by a disturbance): outputs can be generated from the transitivity of the bacterial network, but again could be any other suitable network property. Exemplary species grouped according to their relationship with metabolisms associated with capability to withstand stress conditions include: Exopolysaccharide production capabilities (e.g., with nutrient trapping capabilities, salinity protection capabilities, drought protection capabilities, etc.); heavy metal solubilization (e.g., with bioremediation capabilities, detoxification capabilities, heavy metal stress alleviation capabilities, etc.); salt tolerance capabilities (e.g., with salinity protection capabilities, root growth promotion capabilities, etc.); siderophore production capabilities (e.g., with association iron availability, biofertilizer capabilities, etc.); ACC deaminase capabilities (e.g., with pathogen protection capabilities, with salinity protection capabilities, with drought protection capabilities, etc.); salicylic acid capabilities (e.g., with drought protection capabilities, with salinity protection capabilities, with heavy metal stress alleviation capabilities, etc.); abscisic acid production (e.g., with growth regulation capabilities, with plant resistance capabilities, with yield increase capabilities, etc.).

In examples, low value indices are indicators of aggressive practices, while high value indices are linked to sustainable practices.

As such, generating values of the biosustainability index can include generating a biodiversity value representing species richness, a functionality value representing metagenomic functions, and a resistance value representing stress adaptation of communities represented in the sample dataset.

2.3.2 Selected Effects in Relation to Agronomic Indices related to Health

2. Health: In examples, the invention(s) can generate and implement multiple (e.g., 4) metrics characterizing the role of microorganisms in plant health and yield, as defined by a balance between pathogens, biocontrol agents, and/or other plant growth promotors (e.g., for instance, at an agriculture site input location):

2A. Healthiness (crop health according to detected pathogens): In examples, the invention(s) include steps for generating a score for each disease-risk factor based on the crop-specific pathogen lists. The score (quintile) combines the relative abundance of the disease-risk factor and the resistance score of the soil. Then, based on the quintiles of the minor and major diseases per crop, the invention(s) include architecture for calculating a health score as follows:

At least one major disease in sample at level 5 (maximum quintile), then score=1.

At least one major disease in sample at level 4, then score=2

At least one major disease in sample (not zero), then score=3

At least one minor disease in sample (not zero), then score=4 no disease in sample, then healthiness score=5

In addition to just using the resistance score (i.e., based upon transitivity of networks described above), the invention(s) can further implement transitivity of fungal networks, and co-exclusion proportions in both (bacterial & fungal) networks. Additionally or alternatively, the invention(s) can generate and apply a health index score that is crop-agnostic (i.e. not using disease abundances, but instead, using network properties and principal components of taxonomy and combining them to generate a single score, examples of which are defined above in embodiments, variations, and examples, and further shown in applications incorporated by reference.

In further variations, the invention(s) can be applied to soils known to suppress certain diseases in contrast to soils that allow the diseases to occur, thereby enabling identification of specific taxa or network properties that explain the suppression of the disease pathogens.

In variations, health indices (e.g., soil health indices) can be generated from samples having known management practices (e.g., conventional, organic, biodynamic, etc.), from a wide variety of geographies and crop types (e.g., almond, banana, corn, horticolas, lettuce, mustard, olive, onion, peppers, pimentos, rapeseed, tomatoes, vineyard, wheat, other, etc.). In examples, the dataset generated from samples was split into training and test datasets, and the data was modeled (e.g., using a LASSO Ridge regression, using 16S and ITS data, enriched and depleted, network properties), thereby generating coefficients for modularity, transitivity, assortativity, p-length, and other properties. Coefficients represent the amount by which the health index increases/decreases when a given variable increases by one standard deviation, and can be tagged to indicated interactions between different variables.

Variations of models can include accounting for network properties and principal components from taxonomic annotation (e.g., to improve model fit), where health indices can be divided categorically (e.g., in ranges), characterizing 16S+ITS, 16S only, and ITS only.

In relation to health indices, the method can further include generation of sustainable productivity indices as a proxy for health, where the sustainable productivity indices can be generated as described and/or in applications incorporated by reference.

2B. biocontrol species (microbial species grouped according to the type of pests they encounter, capability of preventing pathogenic species from taking hold or proliferating): The invention(s) can generate relative abundances of the microorganisms on each of these categories: Fungicides, Bactericides, Insecticides, and Nematicides. Additionally or alternatively, the invention(s) can process and apply network properties, since a soil with a high fungal network transitivity and a strong biocontrol set of species is going to be even more resilient to external disruptions (e.g., abiotic, biotic, etc.) than one with just the biocontrol species present but not a high network transitivity.

2C. phytohormone producing species (microbial species grouped according to the type of phytohormone they generate): The invention(s) include steps and architecture for generating relative abundance of microorganisms that produce: Cytokinin production (e.g., with cell proliferation hormone generation, with cell differentiation hormone generation, etc.), Auxin production (e.g., with cell division hormone generation, with stem elongation hormone generation, etc.), and Gibberellin production (e.g., with stem elongation hormone generation, with germination hormone generation, with flowering hormone generation, etc.) for instance, in terms of percentages).

2D. stress sensing and tolerance species (microbial species grouped according to their ability to produce metabolites that help plants withstand stress conditions): The invention(s) include steps and architecture for relative abundance of microorganisms that produce: ACC deaminase, exopolysaccharide production, heavy metal solubilization, salt tolerance, siderophore production, salicylic acid, and abscisic acid.

As such, generating values of the health index can include generating a healthiness value associated with detected pathogens, a biocontrol value representing capability of preventing pathogenic species effects at the agriculture site, a phytohormone value representing generated phytohormones, and a stress value representing metabolites associated with stress withstanding.

2.3.3 Selected Effects in Relation to Agronomic Indices related to Nutrition

3. Nutrition: The invention(s) include steps and architecture for characterizing the potential of soil microorganisms to cycle nutrients and to increase the bioavailability of nutrients for plants, for instance, at an agriculture site input location). Examples of relative abundance of enzymes from predicted metagenomes are described in applications incorporated by reference.

Additionally or alternatively, the inventions can include steps and architecture for processing and applying features related to one or more of:

Carbon (as the basis of soil fertility with release of nutrients for plant growth, promotion of structure and health of soils, and buffer against harmful substances): with identification of new enzyme activities/taxa associated to the potential to sequester carbon. In examples, samples from biodynamic soils (e.g., with no-tilling) with high capacity to sequester carbon, and from traditional soils (e.g. tilling) with low capacity to sequester Carbon, can be processed according to the invention(s) described.

Any nutrient: by determining metabolic fluxes, not just relative abundances of enzyme activities; by determining percentage of enzymes present from a given pathway (not just abundance); by determining function representation in microorganisms from each of the modules in networks. For instance, indices can be related to one or more of: pathways that directly benefit plant nutrition, pathways that take up nutrients from the soil, nitrogen pathways, phosphorus pathways, minor compounds (e.g., sulfur, calcium, chlorine, magnesium, iron, manganese, zinc, copper, and/or other nutrients.

Physicochemical features of any method steps described can be derived from features associated with any or all of the nutrients discussed above and/or other relevant nutrients.

### 2.3.4 Model Architecture Details

Architecture of the model of Step S120 can include neural network model architecture.

The model of Step S120 can additionally or alternatively apply statistical analyses and/or machine learning algorithm(s) can be characterized by a learning style including any one or more of: supervised learning (e.g., using back propagation neural networks), unsupervised learning (e.g., K-means clustering), semi-supervised learning, reinforcement learning (e.g., using a Q-learning algorithm, using temporal difference learning, etc.), and any other suitable learning style. Furthermore, any algorithm(s) can implement any one or more of: a regression algorithm, an instance-based method (e.g., k-nearest neighbor, learning vector quantization, self-organizing map, etc.), a regularization method, a decision tree learning method (e.g., classification and regression tree, chi-squared approach, random forest approach, multivariate adaptive approach, gradient boosting machine approach, etc.), a Bayesian method (e.g., naïve Bayes, Bayesian belief network, etc.), a kernel method (e.g., a support vector machine, a linear discriminant analysis, etc.), a clustering method (e.g., k-means clustering), an associated rule learning algorithm (e.g., an Apriori algorithm), an artificial neural network model (e.g., a back-propagation method, a Hopfield network method, a learning vector quantization method, etc.), a deep learning algorithm (e.g., a Boltzmann machine, a convolution network method, a stacked auto-encoder method, etc.), a dimensionality reduction method (e.g., principal component analysis, partial least squares regression, etc.), an ensemble method (e.g., boosting, boot strapped aggregation, gradient boosting machine approach, etc.), and any suitable form of algorithm.

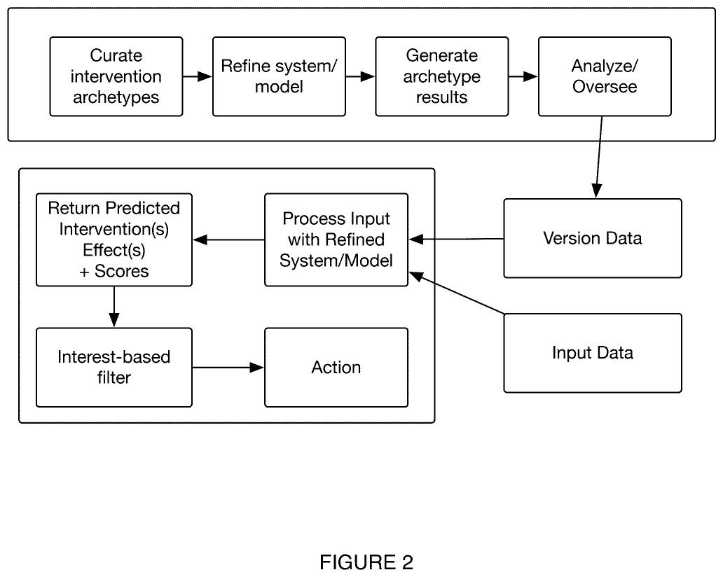

Exemplary model architecture is shown in FIG. 2. In relation to FIG. 2, schema showing phases of the model and applications of use are shown. The initial phase can also be implemented as a version update; first, intervention archetypes are generated. The system is tuned on curated archetypes and results are generated for all archetypes. Additional expert curation is done in a custom application that visualizes system results in a number of settings. If the tuned system passes quality checks, system information (indicated as version data) is submitted to a database so it can be used in different applications. This enables the second phase, where input data is used to predict intervention effects based on the version data. These are then filtered and transformed into actionable insights based on the user's needs.

Alternatively, the model of Step S120 can implement large language model (LLMs) architecture, with respect to the decision support system described herein. LLMs are able to generate text describing agricultural intervention aspects.

Given that LLMs can be prone to hallucinations and may generate information that looks correct but is, in fact, incorrect, training of models incorporating LLM architecture can involve oversight by experts, those with knowledge, or relevant databases of risks associated with agricultural interventions. In particular, outputs of LLM architecture during testing and refinement can be reviewed by entities described in order to identify hazardous interventions and manage their inclusion in decision support outputs, as needed.

LLMs also generally have access to online information and databases, but not to proprietary manufacturer data. As such, LLM architecture of model variations can be trained by manufacturer data and data generated directly from use of various products, where such generated data can be standardized in a manner that prioritizes measurable trial results over marketing claims.

### 2.4 Method-First Variation of Model Refinement

Model refinement in Step S120 can involve refinement based upon improvement or optimization of characteristics for evaluation of model performance, for each effect (e.g., ultimately corresponding to a selected effect during use of the model) resulting from application of an agricultural intervention or archetype of interventions.

In variations, characteristics can include: accuracy values for each archetype and/or agricultural intervention; and sign reliability for each archetype and/or agricultural intervention. In alternative variations, characteristics for evaluating model performance for each archetype and/or individual agricultural interventions can include other metrics of performance.

Model refinement in Step S120 can use intervention archetype data (e.g., with respect to a set of initial intervention archetypes) curated in Step S110, with selection based on the distinctiveness of the data associated with respective archetypes. Use of curated archetypes can avoid weighing a specific kind of intervention too heavily during refinement in Step S120. Alternatively, tuning of model performance can be performed with a weighting process.

Model refinement can be performed in stages, using one or more layers of model architecture, as described herein. In a first variation, using an iterative approach with test data pulled from Step S110, model performance with each iteration of refinement can be evaluated as follows:

With respect to the first characteristic for evaluating model performance, accuracy can be determined from an enumeration of times that the model predicts, from an input location and each agricultural intervention/archetype, a positive intervention effect or a negative intervention effect relative to actually observed intervention effects from the test data. The positive intervention effect and the negative intervention effect can be a composite effect evaluated across all agronomic indices of interest. Alternatively, a positive intervention effect and/or a negative intervention effect can be evaluated for each agronomic index of interest.

With respect to a second characteristic for evaluating model performance, sign reliability can be determined as the fraction of times that the system correctly predicts the sign of the intervention (e.g., in relation to producing a positive effect or a negative effect). The positive intervention effect and the negative intervention effect can be a composite effect evaluated across all agronomic indices of interest. Alternatively, a positive intervention effect and/or a negative intervention effect can be evaluated for each agronomic index of interest.

In addition to performance on observed data, model refinement in Step S120 can involve randomized selection of locations as inputs, with a large number of repetitions, in order to generate null accuracy output data.

In this variation of model refinement in Step S120, accuracy values for each round of iteration are aggregated across all selected intervention archetypes so that a set of predictive features (e.g., optimal predictive features) can be identified that perform well across all archetypes. Predictive features can be associated with network properties, functional annotations, taxonomic data, and/or other data used for labeling, as described in Applications incorporated by reference.

In one example, changes in plant growth-promoting hormone values can be better predicted using microbial metabolism data evaluated in a layer of the model, so the model is able to select the feature(s) only for the effect(s) where it improves overall accuracy. One or more features can be selected at a time, and their weights (e.g., as a measure of importance) in the model are optimized algorithmically. As such, the feature selection process can be individualized per intervention effect, in order to provide the best or most suitable level of accuracy and sign reliability.

Following model refinement in Step S120, the selected sets of features are used to generate results for all archetypes of interest, including those omitted from model refinement. Model refinement in Step S120 thus produces a set of accuracy values (and a set of sign reliability values) per archetype, per intervention effect type, and per set of features. Distances of the features are not comparable, and sign reliability values and distributions of distances can be stored to allow conversion of the model outputs to a comparable format (e.g., with a normalization process).

In this variation, prior to use of the refined model and associated system for generating recommended agricultural interventions from input data in Step S210 and Step S220, model results can be screened (e.g., by an expert) to ensure safety of following recommendations and/or application of agricultural interventions.

### 2.5 Method-Second Variation of Model Refinement

Model refinement in Step S120 can additionally or alternatively involve refinement using other approaches (e.g., a clustering approach, based upon traits for each agricultural intervention or archetype). For instance, refinement can be based upon evaluation of ontologies of agricultural intervention traits. In one example, the trait ‘crop protection’ can be evaluated for various agricultural interventions, and can represent pesticide specificity (e.g. nematicidal activity).

Data can be curated using either unsupervised or supervised approaches.

Data curated in Step S110 can be evaluated for presence or absence of each ontological term, generating a matrix of trait occurrence, using an unsupervised approach. In the second variation of model refinement, hierarchical clustering can be applied to this matrix to identify clusters of ontological terms that co-occur frequently across various agricultural interventions/archetypes. For each cluster, overrepresented traits were selected and used to generate a respective archetype. Archetypes can then be expanded or refined by updating trait occurrence matrices or by adding additional terms to the ontologies.

Data curated in Step S110 can also be curated using a supervised approach. In an example, ontological terms can be evaluated using a supervised knowledge graph approach. Here, ontological terms are represented as a network of connected entities and data available in the proprietary database is curated using the ontological terms as a schema. The resulting graph representation can be queried to obtain archetypes that are both human- and machine-readable. The ontological terms can be dynamically refined and improved by domain experts while maintaining and updating machine-readable representations of the intervention archetypes. Integration of the knowledge graph with the proprietary database can thus enable domain experts to carry out curation at a level of complexity and scale that is otherwise not feasible and enables direct integration with machine learning systems.

Model refinement in the second variation described herein can involve feature extraction/selection with optimization, to process an input (e.g., location, microbiome sample, etc.) and for high performance generation of respective outputs. refinement can also involve an instance-based learning algorithm (e.g., the k-nearest neighbors (KNN)). The instance-based learning algorithm can be used to compare and select the most similar input data categories (e.g., locations, microbiome samples). Each sample is associated with a specific intervention effect, corresponding to a specific product archetype or agricultural intervention.

During model refinement in the second variation, accuracy can be computed per archetype and/or agricultural intervention type, and aggregated across all archetypes and/or agricultural intervention type to generate a measure of overall accuracy. Determination of sign reliability can also be performed for each archetype and/or agricultural intervention type. By computing this separately per archetype/agricultural intervention type, bias is reduced.

In more detail, in the second variation of model refinement, inputs are selected based on the accuracy of the model when only a single input is used. Then, a vector of input weights is generated. This vector can be used to combine the weighted inputs into a distance matrix, for each input of interest. For several iterations, the vector values are optimized and overall accuracy is computed for each vector.

The result of this phase is a list of inputs with associated weights that returned high accuracy across all archetypes/agricultural interventions. The list of inputs determines the inputs that are used to construct the distance matrix to be used in the operation phase, to find the most similar entry.

### 2.6 Method-Practical Applications of Use of Refined Models

Step S210 recites: transforming an input location and a set of selected effects into an agricultural intervention type suited to the input location. While an input location is described, inputs to the refined model can additionally or alternatively include any measure that can be used to describe a sample, and that allows determination of a distance (e.g., a quantitative distance) with respect to data elements. Examples of inputs (e.g. distance) can thus include taxonomic information (e.g. distance: beta diversity), environmental features (e.g. distance: Euclidean distance of environmental characteristics such as soil water content), qualitative metadata such as a crop grown in a location.

In one variation, Step S210 can include processing an input location of interest (e.g., previously-untested input location), where the input location has associated microbiome data and/or additional microbiome data can be generated from the input location with further sampling and testing. Then, for each intervention archetype capable of being evaluated using the model, the model is structured to return values of characteristics, which are then used to assign an intervention effect score for each archetype/agricultural intervention. Step S210 then includes using the sign reliability and distribution of distances generated during use of the model to convert each intervention effect to a level, in order to compare levels/scores to other archetypes. Additionally, Step S210 can include performing a quality control operation. In a specific example, the quality control operation can involve using accuracy values determined upon processing the input, and a null accuracy, to filter any outputs which do not pass a quality control criterion.

Step S210 thus returns a set of predicted intervention effects, based upon the input, where the predicted intervention effects can be used to rank or determine levels of efficacy for each agricultural intervention/archetype. In specific examples, a level can be either negative or positive depending upon the sign of the effect, while its absolute value is based on the system's ability to correctly predict that intervention effect. Levels for different intervention archetypes and effect types can be combined pending on the interest of the user. For example, the best archetype to improve all or selected effects may be returned as the primary model output and recommendation, followed by application of the corresponding agricultural intervention. Alternatively, input data can be a representative collection of locations, providing information on archetype predictions across locations, crops or relevant location characteristics.

During use of the model, only one output archetype/agricultural intervention can be returned; however, more than one output archetype/agricultural intervention can be selected and combined. The proximity of the outputs is calculated using a distance matrix, where the content of the distance matrix is determined during refinement in Step S120. Each sample is associated with a specific intervention effect, corresponding to a specific product archetype. The sample's intervention effect is combined with measures of algorithm performance to create a composite score. The sign of the score therefore represents whether the intervention effect is predicted to be positive or negative, while the scale of the score represents the confidence of the algorithm in this prediction.

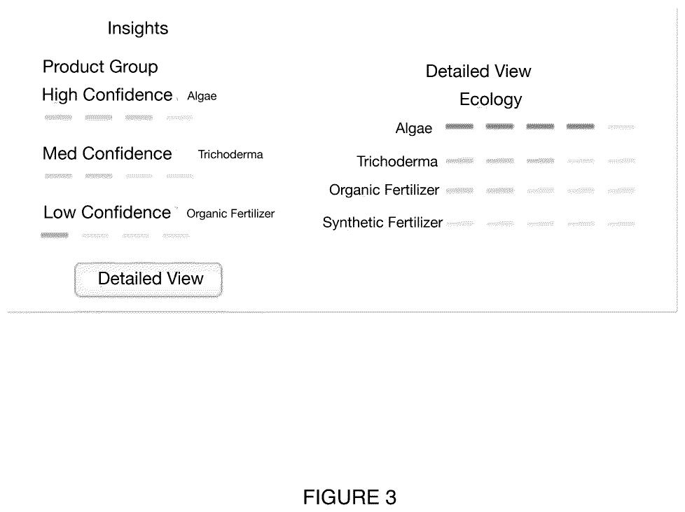

An example of application of the model used according to Step S210 is shown in FIG. 3. FIG. 3 (left) depicts aggregated insights for one location, based on one or multiple microbiome samples collected from that location. FIG. 3 (right) shows a breakdown of the insights into different intervention effects of interest (e.g., e.g. ecology or nutrient status). The exemplary application shown in FIG. 3 provides information on products that could improve yield, health or disease status of the location.

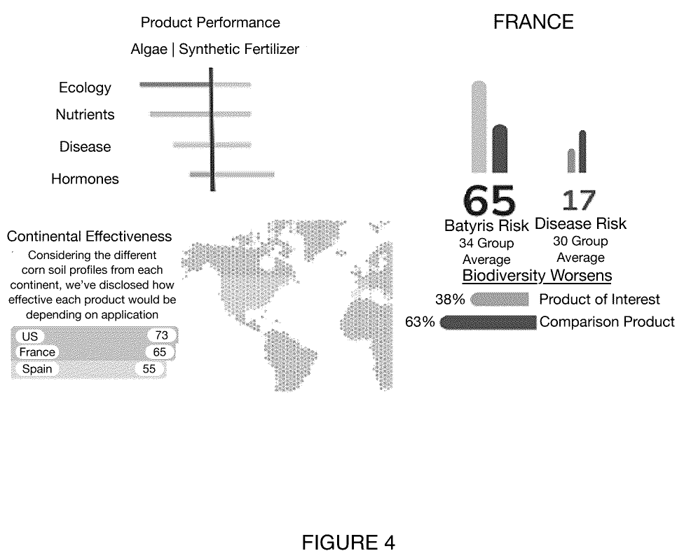

An example application of the model used according to Step S210 is shown in FIG. 4. FIG. 4 (top left) depicts a comparison of two product groups highlighting that fertilizers or biostimulants containing algae may improve ecological status on average relative to synthetic fertilizers. FIG. 4 (right) shows an example of a geographical breakdown of such a comparison, highlighting a specific area where a client's product is predicted to outperform a comparable archetype. The map represents multiple areas.

Step S220 recites: achieving improvement greater than a percentage value with respect to at least one of the selected effects, upon applying the agricultural intervention type at the location. As described above, selected effects in Step S220 can map onto agronomic index values, such that application of a recommended agricultural intervention/archetype to the input location produces an improvement greater than that which could be achieved by other methods.

In variations, the percentage value can be 5%, 10%, 15%, 20%, 35%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, 1000%, 1500%, an intermediate percent, or greater percentages than that described, for each or a combination of different selected effects (e.g., corresponding to agronomic index values, combinations of agronomic index values, etc.).

Relatedly, achieving improvements in relation to step S220 can include achieving improvements in accuracy better than baseline or random predictions attributed to a null model, where the improvements in accuracy can be 5%, 10%, 15%, 20%, 35%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, 1000%, 1500%, an intermediate percent, or greater than baseline or random predictions.

Use Cases and Applications: The methods described can generate insights for any type of agricultural intervention, where samples can be collected and processed to measure and verify the effects of the respective agricultural interventions. Similarly, insights can be extrapolated to a single location or to multiple locations where no interventions have been applied.

A first type of user can apply outputs of the methods described to determine how an agricultural intervention will behave for their specific locations of interest. An exemplary entity corresponding to the first type of user may be an agricultural producer wishing to explore options for managing their plots, or a vendor of agricultural products wishing to provide their clients with personalized advice. In these cases, the input of the system will be samples (e.g., microbiome soil samples) for the location(s) where the agricultural interventions will be applied. Processing inputs according to the methods described can then provide insights into multiple questions, where exemplary questions can relate to product categories that improve nutrition status without harming soil ecology; evaluation of specific products based on its marketing claims (e.g., how do the customized outputs returned by the model suggest it will behave in my location); and other questions. The methods described thus return outputs where associated insights are understandable, actionable and measurable. The insights can also contain information generally unavailable to these clients (e.g. locations where interventions are predicted to perform poorly).

A second type of user can apply outputs of the methods described to generate understanding with respect to how a specific agricultural intervention behaves. An exemplary entity corresponding to the second type of user may be a manufacturer of agricultural products or a vendor of these products. Specific outputs of the methods described can be structured to be different for the second type of user. For example, a manufacturer may be more interested in novel marketing claims, while a vendor may want to use the insights to decide on specific products to stock. In either scenario, the input(s) described can still be processed, and returned outputs can relate to multiple questions related to: evaluations of regions or crops that are more interesting for follow-up research based on hypotheses generated using the refined model; product archetypes that should be used in a comparative study because of predicted improvements of the manufacturer's product over the archetype; additional marketing claims that can truthfully be supported using an independent and data-driven source; recommendations for improving application instructions of the manufacturer's product (e.g., by noting conditions or regions where the system predicts worse performance); and other questions.

For a vendor associated with the second type of user, returned outputs can relate to multiple questions related to: determination of sustainable alternatives that the vendor can recommend for hazardous substances; insights regarding regions or crops where those alternatives may not work well; effects on the soil microbiome that are not listed on the marketing claims for various stocked products, where effects can add value; recommended biologicals with variable performance; and other questions.

In all cases, outputs of the methods described can easily be tailored to construct valuable insights for different users. They are actionable, measurable and simple to understand, supporting their adoption by a wide range of users. Moreover, due to the customizable nature of the outputs, variations of the invention(s) described can improve adoption of agricultural products with variable behaviors by improving user confidence in these products.

### 2.7 Method-Spatial Map Generation

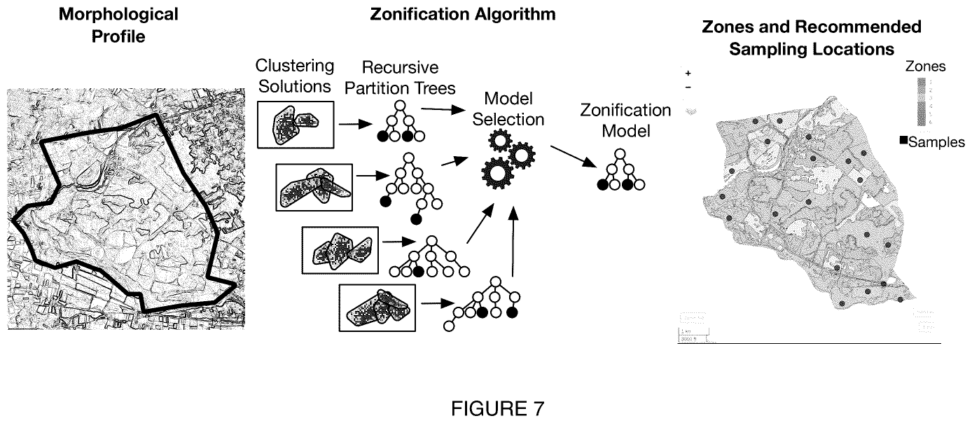

As shown in FIG. 1C, an embodiment of a method 300 includes: generating a spatial map of a set of microbiome features and a set of physicochemical features at an agriculture site, wherein generating the spatial map comprises: receiving a set of samples from a set of recommended sampling sites at the agriculture site, wherein the set of recommended sampling sites is determined from a sampling subsystem structured to generate an analysis of heterogeneity in the agriculture site, and to return the set of recommended sampling sites for the agriculture site upon processing the analysis with remote-sensing and topographic data for the agriculture site S310; generating a mapping predictors catalog from microbiome features and physicochemical features of a set of agriculture sites including the agriculture site S320; and generating the spatial map upon processing samples from the set of recommended sampling sites at the agriculture site along with a second (e.g., supplementary) set of microbiome features and a second (e.g., supplementary) subset of physicochemical features of a subset of samples from the mapping predictors catalog S330. Aspects of system components associated with the method 300 are shown in FIG. 6, and method steps are shown in FIGS. 7 and 9 through 12.

### 2.7.1 Method-Smart Sampling S310

In relation to FIGS. 1C and 6, an embodiment of a smart sampling subsystem used to generate recommended sampling locations for an agriculture site receives, as inputs, 1) an agriculture site morphological profile and b) a set of high-resolution remote-sensing and topographical features. Upon processing the morphological profile and the set of remote-sensing and topographical features, the smart sampling subsystem returns a set of recommended sampling locations that an operator of the agriculture site can retrieve samples (e.g., soil samples, other samples) from (e.g., in relation to Step S310). High-resolution remote-sensing and topographical features can be extracted from detailed imagery and elevation models, thereby enabling precise analysis of the earth's surface features and processes, (e.g., landslides, hillslope processes, urban modeling, etc.) at locations of interest. In examples, high-resolution remote sensing involves acquiring detailed imagery from a distance, typically with spatial resolution of 1-5 meters per pixel or even less than 1 meter per pixel (i.e., very high spatial resolution). In examples, high-resolution topography refers to detailed elevation models of the earth's surface, often obtained through techniques, such as Light Detection and Ranging (LiDAR). Observations can be observed with regard to the electromagnetic spectrum, with passive instruments that use energy from the Sun, with active instruments that provide their own energy sources, and/or other instruments. Resolutions can be 2-bit, 4-bit, 8-bit,16-bit, or another suitable resolution.

In order to achieve the goal of minimizing sampling effort while maximizing sample representativity, in variations, the smart sampling subsystem associated with step S310 is configured to perform:

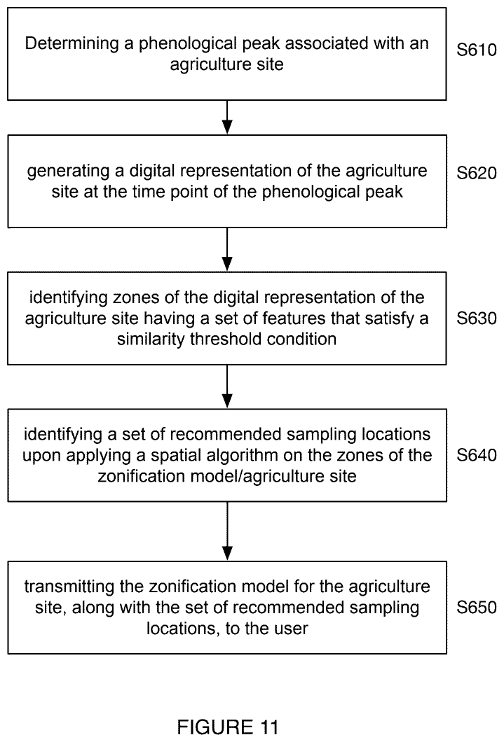

Determining a phenological peak (e.g., the latest phenological peak of a set of crops) at the agriculture site S610, as shown in FIG. 11, wherein the phenological peak represents a point in time during an organism's life cycle where a specific event (e.g., flowering, leafing, or migration) reaches its highest intensity or frequency. In examples, the smart sampling subsystem comprises architecture for building a time series of vegetation index data over a period of time. In embodiments, the vegetation index is a number that quantifies vegetation biomass and/or plant vigor for each pixel and/or group of pixels in a remote sensing image (e.g., using spectral bands sensitive to metrics of plant health). In a specific example, the vegetation index is based upon a Normalized Difference Vegetation Index (NDVI), which compares reflectance in the red and near-infrared regions.

Building the time series of vegetation index data can be performed based upon imaging the agriculture site, imaging sites in proximity to the agriculture site, retrieving data from sensors (e.g., image sensors, thermal sensors, other sensors) at and/or near the agriculture site, and/or by other suitable means. Imaging can be satellite-acquired, aircraft-acquired, drone-acquired, and/or retrieved in another suitable manner. The period of time can be a period on the scale of: one year, two years, three years, four years, an agricultural season, or other period of time. In an example, the smart sampling subsystem 510 generates a low-resolution vegetation index time series over a period of the immediately preceding year, and computes the date of the latest phenological peak, based upon a maximum intensity of a value of the vegetation index.

Upon determining the phenological peak from the time series of vegetation index data, the smart sampling subsystem then performs: generating a digital representation of the agriculture site at the time point of the phenological peak (e.g., the most recent phenological peak from the time series of vegetation index data) S620, as shown in FIG. 11. In a specific example, the smart sampling subsystem comprises architecture for utilizing the high-resolution remote sensing and topographic data inputs to generate a set of raster variables representing the state of the agriculture site, for each unit (e.g., pixel, voxel, other imaging unit) of the agriculture site (e.g., based upon the morphological profile).

Then, the smart sampling subsystem performs: identifying zones of the digital representation of the agriculture site having a set of features that satisfy a similarity threshold condition S630 (as shown in FIGS. 7 and 11), by applying a machine-learning clustering algorithm to the digital representation. In FIG. 7, the left panel depicts a digital representation of an agriculture site, and the middle panel depicts a set of returned outputs of the machine-learning clustering algorithm, wherein the set of returned outputs includes a set of clustering solutions. A set of recursive partition trees corresponding to the set of clustering solutions are evaluated using a model selection process, and the best clustering solution is selected as a zonification model for the agriculture site. The right panel of FIG. 7 depicts a set of zones from the zonification model, where each zone falls within a range of similarity of the similarity threshold condition. The set of features against which the similarity threshold condition is evaluated can include one or more of: location features, vegetation index features, topographical features, environmental features (e.g., an amount of light per day, an amount of shade per day, temperature, pressure, humidity, moisture, elevation, crop type, soil type, etc.). In examples, clustering algorithms implemented by the smart sampling subsystem can include one or more of: centroid-based clustering algorithms, density-based clustering algorithms, hierarchical clustering algorithms, and distribution-based methods, each with algorithms like K-Means, DBSCAN, Agglomerative Clustering, and Gaussian Mixture Models.

The zonification model can be modified/adapted based upon inputs received from an operator of the agriculture site. For instance, the operator can provide defined subregions of the agriculture site, which can be used to adjust boundaries or otherwise redefine zones of the zonification model of the agriculture site.

Upon identifying zones of the agriculture site, the smart sampling subsystem then performs: identifying a set of recommended sampling locations upon applying a spatial algorithm on the zones of the zonification model/agriculture site S640. In order to transform the set of zones of the agriculture site into the set of recommended sampling locations, the smart sampling subsystem 510 applies a sampling algorithm that evaluates features of each zone, where features can include one or more of: geometry of a zone (e.g., total area of the zone), representativity of a zone within the agriculture site (e.g., if the zone is similar to other zones within the agriculture site), area of the agriculture site, a distance of the zone to a border of the agriculture site, and other features.

Upon determining the set of recommended sampling locations, the smart sampling subsystem is then configured to perform: transmitting the zonification model for the agriculture site, along with the set of recommended sampling locations, to the user S650, thereby guiding optimized sampling at the agriculture site.

### 2.7.2 Method-Mapping Predictors Catalog S320

Step S320 recites: generating a mapping predictors catalog from remote-sensing features and topographical featuresof a set of agriculture sites including the agriculture site. System architecture and aspects of the mapping predictors catalog subsystem are described in relation to system elements 520, 521, and 522.

### 2.7.3 Method-Mapping System and Interface S330

Step S330 recites: generating the spatial map upon processing samples from the set of recommended sampling sites of the agriculture site along with a second set of microbiome features and a second subset of physicochemical features of a subset of samples from the mapping predictors catalog. Step S330 can be performed by an embodiment, variation, or example of the mapping subsystem described in relation to system elements 530 and 540 below.

In more detail, embodiments, variations, and examples of Step S330 can involve Steps S710 through S770 (as shown in FIGS. 9, 10, and 12):

Generating a digital representation (e.g., second digital representation) of the agriculture site S710: Generating the digital representation of the agriculture site can include using the morphological profile of the agriculture site and the collection date(s) (i.e., collection time information) of the set of samples for the agriculture site as reference to generate the digital representation. In an example, the digital representation of the agriculture site can be divided into units (e.g., of approximately 10 meters by 10 meters, of approximately another unit area less than 10m×10m, of approximately another unit area more than 10m×10m, etc.). Each of the units/pixels for a unit contains the values of the spectral indices and topographic values for predictions generated by the machine learning models of the mapping subsystem described below.

Generating a training data set from samples of the set of recommended sampling locations and a subset of samples from the mapping predictors catalog subsystem S720: According to step S720, data from samples of the agriculture site and data from a subset of relevant samples from the mapping predictors catalog subsystem are pooled together to generate a training dataset. In examples, the subset of relevant samples can be selected based upon location, time, and crop type, in relation to location(s), time points, and crop types of samples from the set of recommended sampling locations for the agriculture site. In the training dataset, microbiome scores (e.g., from various agronomic indices) and physicochemical scores are the output or response variables, and the remote-sensing and topographic variables (e.g., similar or identical to available variables in the digital representation of the agriculture site) are the input or predictors. As such, the machine learning models (which can include embodiments, variations, and examples of model types described above), can process, as inputs, remote-sensing and topographic variables, and return microbiome index scores and physicochemical feature values for each unit/pixel of the agriculture site.

The training dataset can be iteratively updated whenever incoming data from samples acquired from recommended sampling locations is received. However, the training dataset can be iteratively updated in another suitable manner. Furthermore, the training dataset can be adjusted to include different percentages of data from samples generated using the smart sampling system and data from samples in the mapping predictors catalog subsystem (that were acquired without the smart sampling subsystem). Additionally or alternatively, the weights of data from samples from the smart sampling subsystem can be adjusted relative to the weights of data from samples not acquired using the smart sampling subsystem.

Training a set of ensemble models comprising statistical and machine learning architecture, for each microbiome score category (e.g., a set of microbiome features) and physicochemical score category (e.g., a set of physicochemical features) S730: For each microbiome index (e.g., agronomic index) and physicochemical index, a different ensemble model mixing statistical and machine learning methods is trained (e.g., iteratively trained, whenever incoming data from samples acquired from recommended sampling locations of the smart sampling subsystem is received). During training, the respective ensemble model selects the subset of remote-sensing and topographic predictors with the highest predictive power, where the predictive power can be determined based upon a comparison between predicted values of indices and actual values of indices at various test locations. As such, Step S730 can involve training an ensemble model for each feature (e.g., microbiome, physicochemical, etc.) to return a distribution of microbiome index values and physicochemical index values across a set of units (e.g., positions, pixels) associated with an input location (e.g., agriculture sites of interest).

Generating a prediction map from the trained set of ensemble models S740: In Step S740, each of the trained set of ensemble models is interrogated to determine microbiome index values and physicochemical index values for each unit/pixel of the digital representation of the agriculture site, thereby generating a predictive map for each index, across the digital representation of the agriculture site (e.g., even though samples were acquired from only a subset of locations in the agriculture site).

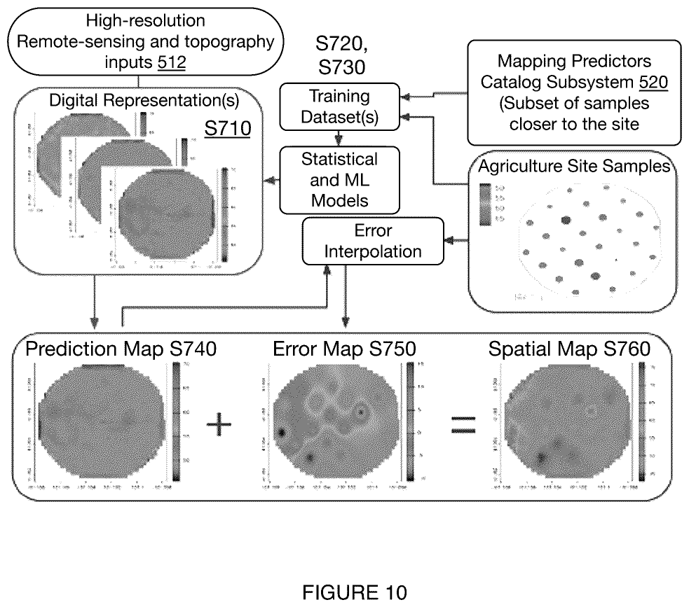

Generating an error map S750 in order to correct prediction errors in the prediction map: In order to correct prediction errors in the prediction map, differences between the pixel values of the prediction map (e.g., for each agronomic index and for each physicochemical index) and the observed values acquired directly from sample data for different units of the digital representation of the agriculture site are interpolated in space to generate an error map, as shown in FIG. 10.

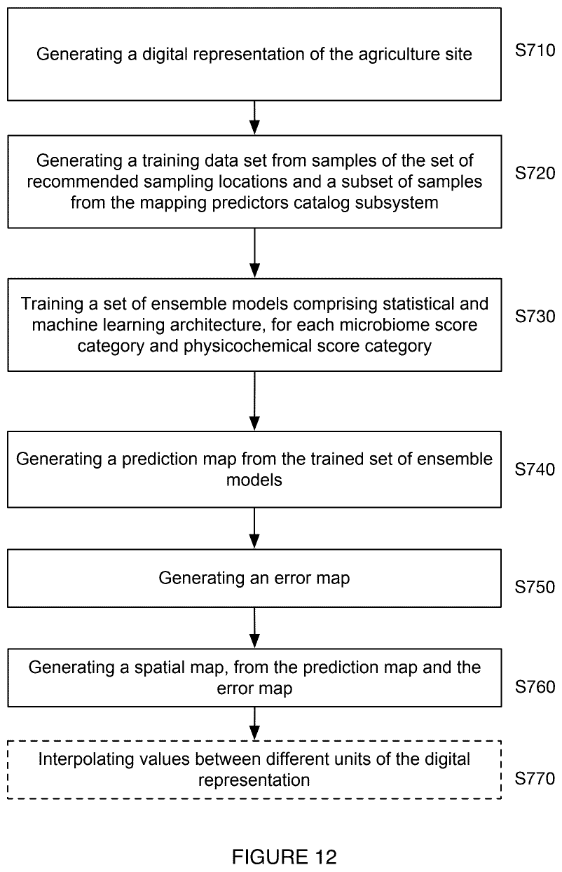

Generating a spatial map, from the prediction map and the error map S760: As shown in FIGS. 10 and 12, co-processing data from the error map and the prediction map (e.g., by adding values of the error map to the prediction map) to cancel out prediction errors is performed, to generate a resultant spatial map for the agriculture site, where the resultant spatial maps depicts, within the user interface, values of agronomic indices and physicochemical indices, for each available location of the digital representation of the agriculture site.

Interpolating values between different units of the digital representation S770: In variations, when performance thresholds are not satisfied for different indices, (e.g., disease indices), the mapping subsystem implements a field interpolation that involves interpolating index values for “unsatisfactory” regions of a digital representation of the agriculture site.

The user interface 540 of the mapping subsystem 530 can be structured to receive inputs (e.g., through input devices) and render outputs (e.g., through output devices). In variations, the user interface 540 can can include an input interface (e.g., touch screen, keyboard, mouse, button, key, microphone, text box, touch input device, sound input device, optical sensor, etc.), which can function to receive input from a user (e.g., agriculture site operator). The input interface can be rendered at a display of a user device, provided as a physical input device, provided as part of an audio input device (e.g., the user device, microphone associated with speech-to-text software, etc.), include any combination of devices, and/or include any other device(s). In examples, the user interface can include one or more of: a computer, a headset (e.g., a virtual reality (VR) headset, an augmented reality (AR) headset, etc.), a mobile device (e.g., smartphone), and/or any other suitable device. Components of a user device can include a display subsystem (e.g., monitor, screen, projected image, etc.), an input subsystem (e.g., keys, touchscreen, microphone, etc.), one or more sensors (e.g., inertial measurement units, accelerometers, gyroscope, cameras, etc.), a processing subsystem, and/or any other suitable subsystem.

### 3. System

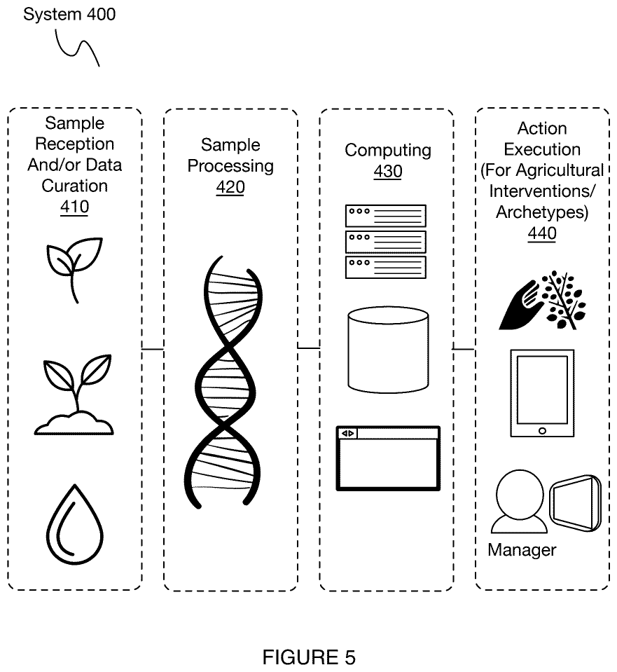

As shown in FIG. 5, a system 400 for characterization and improvement of an agricultural site includes: one or more sample reception subsystems 310; one or more sample processing subsystems 420 in communication with the sample reception subsystems 410; a computing platform 430 comprising one or more processing subsystems comprising non-transitory computer-readable medium comprising instructions stored thereon, that when executed by the processing subsystems perform one or more steps of methods described above; and one or more action execution subsystems 340 configured to execute actions informed by processes of the computing platform 330. In variations, the action execution subsystems 440 can be configured to execute control instructions generated by the computing platform 430, where control instructions can involve instructions for controlling operation modes of one or more of: watering subsystems (e.g., in relation to water distribution through conduits and/or sprinklers to the agriculture site(s)); product delivery subsystems in communication with watering subsystems (e.g., delivery subsystems in communication with watering subsystems through fluidic components, valves, etc.); robotic crop handling subsystems (e.g., in relation to removal of pathogen-affected crop portions); robotic crop picking subsystems (e.g., in relation to automated harvesting at optimal time periods in relation to improving production, in relation to efficiency of new production generation post-harvesting, in relation to minimization of wasted product, etc.); robotic nutrient delivery subsystems (e.g., in relation to initiating delivery, in relation to stopping delivery, in relation to adjusting frequency of delivery, in relation to adjusting delivery dosages, etc.);

greenhouse subsystems; temperature control subsystems (e.g., in relation to modes for controlling environmental temperature of the agriculture site, etc.); light control subsystems (e.g., in relation to modes for controlling environmental light of the agriculture site, in relation to transitioning between on and off states, in relation to light spectrum delivered, etc.); gas environment subsystems (e.g., in relation to modes for controlling environmental gas composition of the agriculture site, etc.); humidity control subsystems (e.g., in relation to modes for controlling environmental humidity levels of the agriculture site, etc.); pressure control subsystems (e.g., in relation to modes for controlling environmental pressure of the agriculture site, etc.); and other suitable subsystem(s) of the agriculture site(s).

Embodiments of the system 400 are configured to perform one or more portions of methods described above; however, variations of the system 400 can be configured to perform other suitable methods. The system 400 can further include elements described in one or more of: U.S. application Ser. No. 17/119,972 filed on 11 Dec. 2020; U.S. application Ser. No. 17/587,016 filed on 28 Jan. 2022; U.S. Application No. 176/665,332 filed on 4 Feb. 2022; and U.S. application Ser. No. 17/703,095 filed on Mar. 24, 2022, each of which is incorporated herein in its entirety by this reference.

### 3.1 System-Smart Sampling and Mapping

As shown in FIG. 6, a system 500 associated with system 400 described above can include: a smart sampling subsystem 510; a mapping predictors catalog subsystem 520; a mapping subsystem 530 comprising a mapping interface 540 structured to render a spatial map of an agriculture site to a user, wherein the spatial map depicts a distribution of a set of microbiome features and a set of physicochemical features across the agriculture site. The system functions to generate high-quality spatial maps that inform end users regarding characteristics of their agriculture sites based on a limited number of physically-retrieved soil samples (or other samples), remote-sensing and topographical data, a catalog of mapping predictors, and other contextual data. One or more components of the mapping system 500 can be provided as a computing system including one or more: central processing units (CPUs), graphics processing units (GPUs), custom field programmable gate array (FPGA)/Application-specific integrated circuits (ASICS), neural processing units (NPUs), processors, microprocessors, servers, cloud computing, storage; memory; and/or any other suitable components. The computing system can be local (e.g., as a local computing system), remote (e.g., as a remote computing system), distributed, or otherwise arranged relative to any other system or module. The components of the computing system can include instructions stored in a non-transitory medium, that when executed, perform one or more steps of the methods 100, 200, 300 described.

In variations, outputs of the mapping system 500 can be used to generate agricultural intervention archetypes suited to a set of selected effects and an input location (e.g., at the agriculture site), such that one or more of the set of microbiome features and the set of physicochemical features for the input location guides selection and application of an appropriate intervention archetype for the input location. However, outputs of the mapping system 500 and/or any location-specific features (e.g., microbiome features, physicochemical features) for an agriculture site can be used to guide application of other interventions or perform a real-world change that affects performance and health of the agriculture site.

### 3.1.1 Smart Sampling Subsystem

An embodiment of the smart sampling subsystem 510 is structured as an automated software subsystem that combines high-resolution remote sensing and topographic data inputs, and includes architecture for analyzing farm heterogeneity, where the remote sensing and topographic data inputs and heterogeneity analysis are used to return recommended soil sampling locations for the agriculture site. By providing a reduced number of recommended sampling locations, the smart sampling subsystem 510 increases access to the outputs of the mapping subsystem 530 by reducing overall sampling requirements, and while still enabling provision of high-resolution and accurate maps. In examples, the smart sampling subsystem 510 can optimize/significantly reduce soil sampling costs and ensures sample representativity for the mapping subsystem 530. As such, the smart sampling subsystem 510 is designed to minimize sampling effort while maximizing sample representativity.

As shown in FIG. 6, an embodiment of the smart sampling subsystem 510 receives, as inputs, 1) an agriculture site morphological profile 511 and b) a set of high-resolution remote-sensing and topographical features 512. Upon processing the morphological profile 511 and the set of remote-sensing and topographical features 512, the smart sampling subsystem returns a set of recommended sampling locations that an operator of the agriculture site can retrieve samples (e.g., soil samples, other samples) from. In order to achieve the goal of minimizing sampling effort while maximizing sample representativity, in variations, the smart sampling subsystem 510 is configured to perform:

Determining a phenological peak (e.g., the latest phenological peak of a set of crops) at the agriculture site S610, as shown in FIG. 11, wherein the phenological peak represents a point in time during an organism's life cycle where a specific event (e.g., flowering, leafing, or migration) reaches its highest intensity or frequency. In examples, the smart sampling subsystem 510 comprises architecture for building a time series of vegetation index data over a period of time. In embodiments, the vegetation index is a number that quantifies vegetation biomass and/or plant vigor for each pixel and/or group of pixels in a remote sensing image (e.g., using spectral bands sensitive to metrics of plant health). In a specific example, the vegetation index is a Normalized Difference Vegetation Index (NDVI), which compares reflectance in the red and near-infrared regions. However, the vegetation index can additionally or alternatively include another suitable vegetation index.

Building the time series of vegetation index data can be performed based upon imaging the agriculture site, imaging sites in proximity to the agriculture site, measuring pollen indices near and/or at the agriculture site, retrieving data from sensors (e.g., image sensors, thermal sensors, other sensors) at and/or near the agriculture site, and/or by other suitable means. Imaging can be satellite-acquired, aircraft-acquired, drone-acquired, and/or retrieved in another suitable manner. The period of time can be a period on the scale of: one year, two years, three years, four years, an agricultural season, or other period of time. In an example, the smart sampling subsystem 510 generates a low-resolution vegetation index time series over a period of the immediately preceding year, and computes the date of the latest phenological peak, based upon a maximum intensity of a value of the vegetation index.

Upon determining the phenological peak from the time series of vegetation index data, the smart sampling subsystem 510 then performs: generating a digital representation of the agriculture site at the time point of the phenological peak (e.g., the most recent phenological peak from the time series of vegetation index data) S620, as shown in FIG. 11. In a specific example, the smart sampling subsystem comprises architecture for utilizing the high-resolution remote sensing and topographic data inputs to generate a set of raster variables representing the state of the agriculture site, for each unit (e.g., pixel, voxel, other imaging unit) of the agriculture site (e.g., based upon the morphological profile).

Then, the smart sampling subsystem 510 performs: identifying zones of the digital representation of the agriculture site having a set of features that satisfy a similarity threshold condition S630 (as shown in FIGS. 7 and 11), by applying a machine-learning clustering algorithm to the digital representation. In FIG. 7, the left panel depicts a digital representation of an agriculture site, and the middle panel depicts a set of returned outputs of the machine-learning clustering algorithm, wherein the set of returned outputs includes a set of clustering solutions. A set of recursive partition trees corresponding to the set of clustering solutions are evaluated using a model selection process, and the best clustering solution is selected as a zonification model for the agriculture site. The right panel of FIG. 7 depicts a set of zones from the zonification model, where each zone falls within a range of similarity of the similarity threshold condition. The set of features against which the similarity threshold condition is evaluated can include one or more of: spectral indices derived from high-resolution remote sensing data, such as, vegetation indices, soil humidity indices, soil mineralogy indices, and high-resolution topographic indices such as topographic slope. In examples, clustering algorithms implemented by the smart sampling subsystem can include one or more of: centroid-based clustering algorithms, density-based clustering algorithms, hierarchical clustering algorithms, and distribution-based methods, each with algorithms like K-Means, DBSCAN, Agglomerative Clustering, and Gaussian Mixture Models.

The zonification model can be modified/adapted based upon inputs received from an operator of the agriculture site. For instance, the operator can provide defined subregions of the agriculture site, which can be used to adjust boundaries or otherwise redefine zones of the zonification model of the agriculture site.

Upon identifying zones of the agriculture site, the smart sampling subsystem then performs: identifying a set of recommended sampling locations upon applying a spatial algorithm on the zones of the zonification model/agriculture site S640. In order to transform the set of zones of the agriculture site into the set of recommended sampling locations, the smart sampling subsystem 510 applies a sampling algorithm that evaluates features of each zone, where features can include one or more of: geometry of a zone (e.g., total area of the zone), representativity of a zone within the agriculture site (e.g., if the zone is similar to other zones within the agriculture site), area of the agriculture site, a distance of the zone to a border of the agriculture site, and other features.

Upon determining the set of recommended sampling locations, the smart sampling subsystem 510 is then configured to perform: transmitting the zonification model for the agriculture site, along with the set of recommended sampling locations, to the user S650, thereby guiding optimized sampling at the agriculture site.

### 3.1.2 Mapping Predictors Catalog Subsystem

An embodiment of the mapping predictors catalog subsystem 520, shown in FIG. 6, functions to receive, as inputs, 1) high-resolution remote sensing and topographic data inputs, and 2) samples and/or data from samples associated with the set of recommended sampling locations generated from Step S640 performed by the smart sampling subsystem. The mapping predictors catalog subsystem 520 is structured as an automated software and database system that links high-resolution remote-sensing and topographic data with data from all soil samples of the platform described. The mapping predictors catalog subsystem 520 also functions to provide the system with robust training data, where the training data is iteratively expanded with new incoming samples/sample data from newly evaluated agriculture sites. As such, the mapping predictors catalog subsystem provides high-quality training data to increase robustness of models of the system (e.g., smart sampling subsystem 510, mapping subsystem 530), while reducing the number of samples required to map a given agriculture site.

In embodiments, the mapping predictors catalog subsystem 520 comprises a specialized software portion 521 and a specialized database portion 522. In embodiments, the software portion 521 comprises automatic, constantly-running architecture structured to associate data from all incoming soil samples with high-resolution remote sensing and topographic data (e.g., by coordinates, by collection time). The specialized software portion 521 performs these steps with every group of new samples collected, thereby accumulating longitudinal sample data across all agriculture sites, all sampling locations, and all sample acquisition dates. The specialized software portion also retrieves high-resolution remote sensing data, composes a cloudless scene, and computes spectral indices (e.g., associated with vegetation indices of the smart sampling subsystem 510) used in the mapping subsystem 530. Finally, the software portion 521. The pixel values at the sample locations for the spectral and topographic variables are organized, by the software portion 521 (e.g., as a table) and written to the specialized database portion 522 of the mapping predictors catalog subsystem 520.

As shown in FIG. 8, an exemplary representation of the database portion 522 of the mapping predictors catalog subsystem 520 is structured to store data (e.g., pixel values at the sample locations for the spectral and topographic variables) from the software portion 521, to normalize the data, to control data quality, and to transmit data to the mapping subsystem 530 when interrogated by the mapping subsystem 530.

Data in the database portion 522 of the mapping predictors catalog subsystem 520 can be organized according to a schema, as shown in FIG. 8. Exemplary data features for each location of the agriculture site(s)/pixel(s) include: spectral band wavelengths (e.g., 490 nm, 560 nm, 665 nm, 705 nm, 740 nm, 783 nm, 842 nm, 1610 nm, 2190 nm) acquired with different resolution parameters (e.g., 10m, 20m, etc.); normalized differences between different spectral band wavelengths, normalized near infrared (NIR) values; ferric iron (FE2+) values, soil brightness index values (e.g., Misra soil brightness index values); normalized different near infrared/red values; normalized vegetation index values, calibrated NDVI-CDVI values; blue-wide dynamic range vegetation index values, enhanced vegetation index values, moisture stress index values (e.g., ratio 1599/819 values); elevation (e.g., in meters above sea level) values; surface steepness values (e.g., in degrees from 0-90 degrees); topographic position index values; terrain roughness index values; elevation different values (where elevation difference can be determined from values of a pixel in relation to neighboring pixels); direction of water flow values (determined from steepest descent in elevation across pixels); exposure of a slope facing north values, exposure of a slope facing east values; exposure of a slope facing west values; exposure of a slope facing south values; and/or other metric values. Such features can thus augment/expand upon the high-resolution remote sensing and topographic data inputs described.

Data features of database portion 522 of the mapping predictors catalog subsystem 520 can be retrieved according to the organizational schema, where aspects can be organized by satellite metrics (e.g., code, description, name of satellite); location identifiers (e.g., location name, coordinates); environmental predictors based on satellite identifiers and/or satellite metrics; longitude, latitude, date; and/or other predictors.

### 3.1.3 Mapping Subsystem

As shown in FIG. 6, an embodiment of the mapping subsystem 530 receives as inputs, for each of a set of pixels associated with the agriculture site: 1) features retrieved from the mapping predictors catalog subsystem 520, 2) high-resolution remote sensing and topography data outside of the mapping predictors catalog subsystem, 3) scores of agronomic indices described, and 4) the morphological profile of the agriculture site; and returns a spatial map for rendering at a user interface 540 of the mapping subsystem 530. As such, the mapping subsystem functions as an automated software system to generate high-resolution microbiome and physicochemical maps at the scale of the agriculture site (or different scales), by combining data from the samples of the agriculture site (selected by the smart sampling subsystem 510) with a subset of samples from the mapping predictors catalog 520, and the agronomic index scores (e.g., provided using methods and systems of the platform described above). As such, the mapping system generates a spatial map, wherein the spatial map depicts a distribution of the set of microbiome features and the set of physicochemical features across the agriculture site.

In an embodiment, the mapping application is triggered when all required data for a given agriculture site is available, wherein in examples, required data can include one or more of: morphological profile aspects of an agriculture site, sample coordinates, collection dates, physicochemical scores, microbiome feature values of the agronomic indices, and/or other features.

In an example, the mapping subsystem 530 includes architecture for generating a spatial map for the agriculture site, upon performing the following steps (as shown in FIGS. 9,10, and 12):

Generating a digital representation (e.g., second digital representation) of the agriculture site S710: Generating the digital representation of the agriculture site can include using the morphological profile of the agriculture site and the collection date(s) of the set of samples for the agriculture site as reference to generate the digital representation. In an example, the digital representation of the agriculture site can be divided into units (e.g., of approximately 10 meters by 10 meters, of approximately another unit area less than 10m×10m, of approximately another unit area more than 10m×10m, etc.). Each of the units/pixels for a unit contains the values of the spectral indices and topographic values.

Generating a training data set from samples of the set of recommended sampling locations and a subset of samples from the mapping predictors catalog subsystem S720: According to step S720, data from samples of the agriculture site and data from a subset of relevant samples from the mapping predictors catalog subsystem are pooled together to generate a training dataset. In examples, the subset of relevant samples can be selected based upon location, time, and crop type, in relation to location(s), time points, and crop types of samples from the set of recommended sampling locations for the agriculture site. In the training dataset, microbiome scores (e.g., from various agronomic indices) and physicochemical scores are the output or response variables, and the remote-sensing and topographic variables (e.g., similar or identical to available variables in the digital representation of the agriculture site) are the input or predictors. As such, the machine learning models (which can include embodiments, variations, and examples of model types described above), can process, as inputs, remote-sensing and topographic variables, and return microbiome index scores and physicochemical feature values for each unit/pixel of the agriculture site.

The training dataset can be iteratively updated whenever incoming data from samples acquired from recommended sampling locations is received. However, the training dataset can be iteratively updated in another suitable manner. Furthermore, the training dataset can be adjusted to include different percentages of data from samples generated using the smart sampling system and data from samples in the mapping predictors catalog subsystem (that were acquired without the smart sampling subsystem). Additionally or alternatively, the weights of data from samples from the smart sampling subsystem can be adjusted relative to the weights of data from samples not acquired using the smart sampling subsystem.

Training a set of ensemble models comprising statistical and machine learning architecture, for each microbiome score category and physicochemical score category S730: For each microbiome index (e.g., agronomic index) and physicochemical index, a different ensemble model mixing statistical and machine learning methods is trained (e.g., iteratively trained, whenever incoming data from samples acquired from recommended sampling locations of the smart sampling subsystem is received). During training, the respective ensemble model selects the subset of remote-sensing and topographic predictors with the highest predictive power, where the predictive power can be determined based upon a comparison between predicted values of indices and actual values of indices at various test locations. In examples, one or more of the ensemble models can include one or more of: a generalized additive model (GAM), a generalized linear model (GLM), a random forest (RF) model, a simple interpolation model, a lookup table, and/or another form of model architecture.

Generating a prediction map from the trained set of ensemble models S740: In Step S740, each of the trained set of ensemble models is interrogated to determine microbiome index values and physicochemical index values for each unit/pixel of the digital representation of the agriculture site, thereby generating a predictive map for each index, across the digital representation of the agriculture site (e.g., even though samples were acquired from only a subset of locations in the agriculture site).

Generating an error map S750 in order to correct prediction errors in the prediction map: In order to correct prediction errors in the prediction map, differences between the pixel values of the prediction map (e.g., for each agronomic index and for each physicochemical index) and the observations acquired directly from sample data for different units of the digital representation of the agriculture site are interpolated in space to generate an error map, as shown in FIG. 10.

Generating a spatial map, from the prediction map and the error map S760: As shown in FIGS. 10 and 12, co-processing data from the error map and the prediction map (e.g., by adding values of the error map to the prediction map) to cancel out prediction errors is performed, to generate a resultant spatial map for the agriculture site, where the resultant spatial maps depicts, within the user interface, values of agronomic indices and physicochemical indices, for each available location of the digital representation of the agriculture site.

Interpolating values between different units of the digital representation S770: In variations, when performance thresholds are not satisfied for different indices, (e.g., disease indices), the mapping subsystem implements a field interpolation that involves interpolating index values for “unsatisfactory” regions of a digital representation of the agriculture site.

The user interface 540 of the mapping subsystem 530 can be structured to receive inputs (e.g., through input devices) and render outputs (e.g., through output devices). In variations, the user interface 540 can can include an input interface (e.g., touch screen, keyboard, mouse, button, key, microphone, text box, touch input device, sound input device, optical sensor, etc.), which can function to receive input from a user (e.g., agriculture site operator). The input interface can be rendered at a display of a user device, provided as a physical input device, provided as part of an audio input device (e.g., the user device, microphone associated with speech-to-text software, etc.), include any combination of devices, and/or include any other device(s). In examples, the user interface can include one or more of: a computer, a headset (e.g., a virtual reality (VR) headset, an augmented reality (AR) headset, etc.), a mobile device (e.g., smartphone), and/or any other suitable device. Components of a user device can include a display subsystem (e.g., monitor, screen, projected image, etc.), an input subsystem (e.g., keys, touchscreen, microphone, etc.), one or more sensors (e.g., inertial measurement units, accelerometers, gyroscope, cameras, etc.), a processing subsystem, and/or any other suitable subsystem.

### 3.2 System-Smart Sampling and Mapping

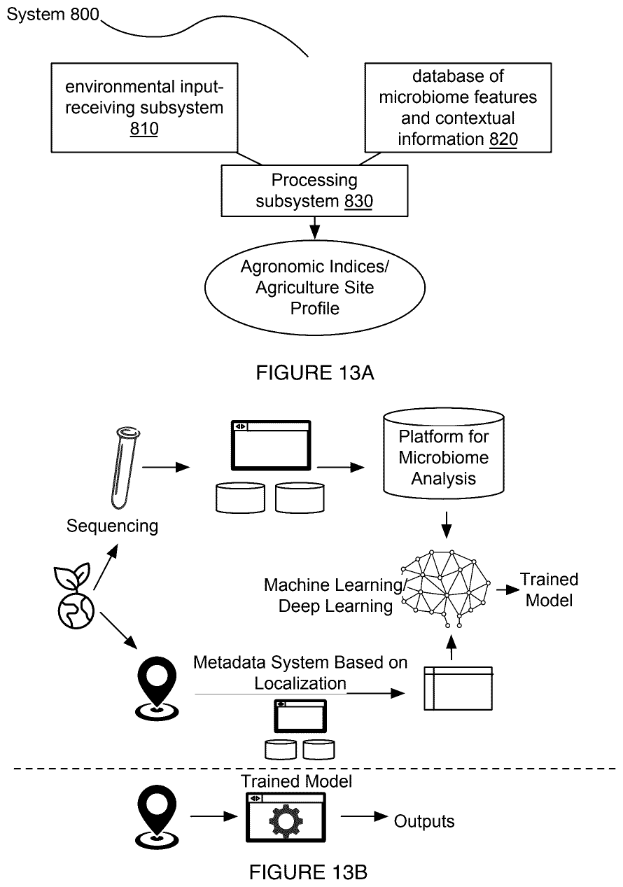

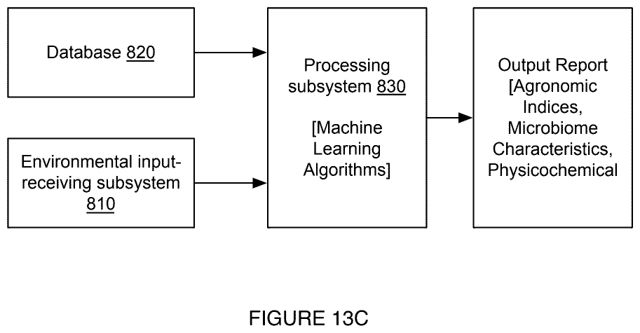

As shown in FIGS. 13A, 13B, and 13C, a system 800 for predicting microbiome composition includes: an environmental input-receiving subsystem 810; a database of microbiome features and contextual information 820; and a processing subsystem 830 comprising instructions stored in a non-transitory computer-readable medium that, when executed, perform: receiving an input from the environmental input-receiving subsystem; and returns a set of agronomic indices corresponding to the input, upon processing the input with information from the database 820. As such, the system 800 is able to estimate agronomic indices described above, from environmental information. The system 800 can infer microbiome indices profiles for a particular set of coordinates (e.g., latitude, longitude) based solely on environmental information by leveraging the database 820. Additional inputs received from the environmental input-receiving subsystem can include one or more of: crop type details, management practice information (e.g., organic, conventional, etc.), physicochemical measurements, and/or other contextual information associated with samples.

The system 800 functions to execute embodiments, variations, and examples of computational method for predicting microbiome composition from cheaper and more easily-measured features. The system 800 is configured to provide a scalable solution with development of scalable, accessible, and effective computational approaches for microbial profiling to aid decision-making in the smart farming field, extending the accessibility of soil microbiome technologies to a wider public. Additional motivation for the described technology solution: soil microorganisms are major drivers of terrestrial biogeochemistry, taking part in a myriad of processes affecting soil. Therefore, this knowledge is crucial in agriculture to ensure soil efficiency, yield, and health. Despite the enormous advances that microbiome technology has experienced in recent years, microbiome studies still involve technical requirements at a certain economical cost; in particular, the access to sequencing facilities. Thus, not all sites have access and resources for sequencing (such as emerging countries), or they may be too costly in some cases (even in developed countries when experimental designs are big).

Exemplary Novel Aspects: the system 800 and associated methods provide low-cost soil profiling; use of environmental data to predict corresponding microbiome profiles and/or agronomic indices; obviating a need for in situ sampling; worldwide usage (e.g., in accordance with an “environmental reduced space”); identification of where predictions are appropriate (Area of Applicability); provision of access to microbiome advances in regions with a lack of sequencing facilities; and other novelties.

The system 800 leverages, in a novel manner, the following:
1) The database 820, which includes sequences acquired from samples from agriculture sites, and corresponding agronomic indices. The database 820 stores the results of the samples processed according to laboratory methods described in applications incorporated by reference, in order to generate corresponding agronomic indices.
2) Contextual environmental information for cultivable areas globally, (e.g., extracted and/or derived from metadata infrastructure).
3) Contextual environmental information for samples obtained agriculture sites and/or metadata infrastructure.

In embodiments, variations, and examples, the metadata infrastructure provides a set of tools that process publicly available satellite information from several providers and stores the satellite information for further usage (e.g., in relation to characterizing cultivable areas, in relation to characterizing samples).

By pairing the database 820 and the metadata infrastructure, the system 800 constructs a dataset that associates environmental descriptors of a sample with agronomic indices. Next, this dataset is depurated to eliminate missing data, overlapping data, and extreme values. Additionally or alternatively, the dataset can be processed and/or transformed for other downstream uses.

In parallel, global environmental data from a set of points (e.g., 49 million points) from the metadata infrastructure, corresponding to crop areas, are reduced (e.g., to 9 million points) to a smaller set that represents the global variation in the descriptors. Reducing the global environmental data from the set of points to a reduced subset can implement a process that reduces the dimensionality of the “environmentally reduced space”. This process thus provides a comparable way to compute an “environmental distance” between any sample point. Reduction and/or distance/similarity characterizations can, however, be performed in another suitable manner.

In embodiments, variations, and examples, the depurated dataset is then processed by the processing subsystem 830, which comprises architecture for a machine learning model that is trained to predict agronomic indices from environmental variables. In embodiments, different machine learning algorithms can be used to train the architecture of the processing subsystem 830, including but not limited to: Linear Models, Generalized Additive Models, Random Forest, XGBoost, and Neural Networks (e.g., foundation models). Other forms of machine learning architecture and training methods that can be used are described above and/or in Applications incorporated by reference. Then, using the “environmental reduced space” model described, the processing subsystem 830 is structured to determine which areas are within the prediction range of the model, which is called the “Area of Applicability” (AoA), and to inform an entity (e.g., end user) about the confidence of this value related to the environmental distance of a target point to associated samples. Using the AoA, the system 800 can also generate rough estimates of a number of samples recommended to reliably predict a new region not previously covered by the system 800.

In a specific example, training of the models of the system 100 involves training using spatial data. In more detail, the following approach can be used:

Data is divided into blocks, considering the climatic diversity structure (e.g., environmental zones, as delivered by an embodiment, variation, or example of the zonification algorithm described above). In the specific example, one block is kept separate from the training dataset and used to estimate the error of the method. Next, in the specific example, another block of data is used for error estimation while the previous block is incorporated into the training data. This process is repeated for every block in the specific example. In the assessment procedure, the amount of blocks for training and testing could change. With this, an error estimation of the method is obtained.

FIG. 13B depicts an exemplary flow of a method associated with system 800 described above. In FIG. 13B, samples are collected and stored in the database 820, and agronomic indices are calculated to be stored in a report. In parallel, based on location, environmental information is collected in coordination with the environmental input-receiving subsystem 810, and paired with data involving the agronomic indices. Finally, algorithm is trained with this data. Then, the trained model is applied to one or multiple virtual samples by inputting its environmental descriptors to obtain a predicted low-cost map or report of each of a set of agronomic indices and microbiome characterizations for the agriculture site(s) of interest.

FIG. 13C depicts an application of the invention(s) associated with the system 800. Using the database 820, the environmental data from the environmental input-receiving subsystem 810, and the trained machine learning algorithm architecture of with processing system 830, the invention delivers a substantially-equivalent report associated with each sample for one or more agriculture sites.

### 4. Conclusions

The invention(s) decipher different ecological strategies that bacterial, fungal, and/or other organism communities adopt in face of different levels of farming intensification and product use, and explore how these may impact soil health in terms of external factors and plant pathogens. In applications, outputs of the invention(s) can guide interventions and/or other practices to improve agriculture sites, as observed community assembly strategies. In examples, a collaborative well-mixed habitat in soils under biodynamic management with potentially higher resistance towards, at least, pathogen loads, or a more divided habitat, with fungi belonging to more niches but with lower reaction range to pathogen loads in soils under conventional management. Under this framework, the inventions have practical applications with relevance for agriculture sustainability, and with respect to interventions that can be designed to drive a better future for agro-ecosystems. For instance, evaluating how emergent properties change during time-series, may give clear indications about the resistance and resilience of fungal communities, or shed light into the dynamics of soils under different anthropogenic disturbances. For now, the defined ecological emergent properties may be used as biomarkers to measure the effect of farming practices or temperature change consequences in the health status of soils. Given the key role that microorganisms play in agri-food systems in general, and in crop yield in particular, these findings are useful for establishing monitoring programs of crop-associated microbial diversity, supporting the work of alliances such as the soil health institute the U.S. department of agriculture, or the global initiative of crop microbiome and sustainable agriculture, while promoting soil healthiness through agriculture sustainable strategies.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

What is claimed is:

1. A method for generating a spatial map of an agriculture site, the method comprising:
receiving a set of samples from a set of recommended sampling sites at the agriculture site, wherein the set of recommended sampling sites is determined from a sampling subsystem structured to generate an analysis of heterogeneity in the agriculture site, and to return the set of recommended sampling sites for the agriculture site upon processing the analysis at a phenological peak of the agriculture site;
generating a mapping predictors catalog from remote-sensing features and topographical features of a set of agriculture sites including the agriculture site; and
generating the spatial map upon processing samples from the set of recommended sampling sites at the agriculture site along with a supplementary set of microbiome features and a supplementary subset of physicochemical features of a subset of samples from the mapping predictors catalog.

2. The method of claim 1, further comprising generating the set of recommended sampling sites upon:
determining the phenological peak at the agriculture site; generating a digital representation of the agriculture site at the time point of the phenological peak;
generating a zonification model with identification of a set of zones of the digital representation of the agriculture site having a set of features that satisfy a similarity threshold condition;
identifying the set of recommended sampling locations upon applying a spatial algorithm to the set of zones; and transmitting the zonificaiton model with the set of recommended sampling locations to a user.

3. The method of claim 2, wherein determining the phenological peak comprises evaluating a time series of a vegetation index over a duration of time, and identifying the phenological peak based upon a maximum intensity of a value of the vegetation index during the period of time.

4. The method of claim 2, wherein generating the zonification model comprises transforming the digital representation into a set of clustering solutions, and evaluating a set of recursive partition trees corresponding to the set of clustering solutions with using a model selection process.

5. The method of claim 2, wherein applying the spatial algorithm comprises evaluating a set of parameters for each of the set of zones, wherein the set of parameters comprises: geometry of a zone, representativity of a zone within the agriculture site, and a distance of the zone to a border of the agriculture site.

6. The method of claim 1, wherein the set of samples comprises soil samples.

7. The method of claim 1, wherein the sampling subsystem reduces a number of samples required to generate the spatial map by at least 50% in comparison with a process that omits involvement of the sampling subsystem.

8. The method of claim 1, wherein generating the spatial map comprises:
generating a digital representation of the agriculture site, wherein the digital representation comprises a morphological profile of the agriculture site and collection time information for the set of samples;
generating a training dataset from the set of samples and a subset of samples from the mapping predictors catalog;
for each of a set of microbiome features and a set of physicochemical features, training an ensemble model to return a distribution of microbiome index values and physicochemical index values across a set of units associated with an input location.

9. The method of claim 8, further comprising: iteratively updating the training dataset whenever incoming data from samples acquired from recommended sampling sites, generated using the sampling subsystem, is received.

10. The method of claim 8, further comprising generating a prediction map from the ensemble model for each microbiome index value and each physicochemical index value, across the digital representation of the agriculture site, and generating the spatial map from the prediction map.

11. The method of claim 10, further comprising generating an error map upon determining differences between pixel values of the prediction map and observed values acquired directly from sample data from the set of samples corresponding to the set of recommended sampling sites.

12. The method of claim 1, further comprising rendering the spatial map at a user interface.

13. The method of claim 1, further comprising processing an input location for the agriculture site and a set of selected effects for the agriculture site, transforming the input location and the set of selected effects into an agricultural intervention type suited to the agriculture site input location; and
applying the intervention type at the agriculture site.

14. A system comprising:
a smart sampling subsystem structured to receive 1) an agriculture site morphological profile and b) a set of high-resolution remote-sensing and topographical features and return a set of recommended sampling sites from an agriculture site;
a mapping predictors catalog subsystem structured to catalog a set of remote-sensing features and a set of topographical features of the agriculture site, in response to processing data from a set of samples corresponding to the set of recommended sampling sites from the agriculture site;
a mapping subsystem comprising a mapping interface configured to generate and render a spatial map of the agriculture site to a user, wherein the spatial map depicts a distribution of the set of microbiome features and the set of physicochemical features across the agriculture site and is generated upon interrogating the mapping predictors catalog subsystem.

15. The system of claim 14, comprising instructions stored in a non-transitory medium, that when executed, perform: determining a phenological peak at the agriculture site;
generating a digital representation of the agriculture site at the time point of the phenological peak;
generating a zonification model with identification of a set of zones of the digital representation of the agriculture site having a set of features that satisfy a similarity threshold condition;
identifying the set of recommended sampling locations upon applying a spatial algorithm to the set of zones; and
transmitting the zonification model with the set of recommended sampling locations to a user.

16. The system of claim 14, comprising instructions stored in a non-transitory medium, that when executed, perform generating the spatial map, wherein generating the spatial map comprises:
generating a digital representation of the agriculture site, wherein the digital representation comprises the agriculture site morphological profile and collection time information for the set of samples;
generating a training dataset from the set of samples and a subset of samples from the mapping predictors catalog subsystem;
for each of the set of microbiome features and the set of physicochemical features, training an ensemble model to return a distribution of microbiome index values and physicochemical index values across a set of units associated with the agriculture site.

17. A method comprising:
generating a dataset pertaining to a set of agricultural interventions, wherein a data element for an agricultural intervention of the set of agricultural interventions comprises: a set of location characteristics corresponding to a location at which the agricultural intervention will be applied, at a first time point, and an effect of the agricultural intervention at the location at a second time point;
iteratively refining a model that transforms input locations and selected effects into a returned agricultural intervention archetypes suited to the selected effects and the input locations, wherein refining the model comprises training the model with the dataset along with a set of performance criteria;
receiving an agriculture site input location and a set of selected effects for the agriculture site;
with the model, transforming the agriculture site input location and the set of selected effects into an agricultural intervention type suited to the agriculture site input location; and
improving performance related to the set of selected effects at the agriculture site input location, upon applying the agricultural intervention type at the agriculture site input location, wherein performance is evaluated in relation to a null model and changes in a set of agronomic index values at the agriculture site input location.

18. The method of claim 17, wherein the agricultural intervention type comprises at least one of a pesticide, a fertilizer, a biostimulant, and a management practice.

19. The method of claim 17, wherein the set of selected effects comprises a yield effect, a biodiversity effect, and a biosustainability effect.

20. The method of claim 17, wherein the set of agronomic index values comprises:
a biosustainability index characterizing diversity of sample species and metabolic functions of a microbiome associated with the agriculture site input location,
a health index characterizing pathogens and disease risk of a microbiome associated with the agriculture site input location; and
a nutrition index characterizing potential of microorganisms to cycle nutrients and to increase the bioavailability of nutrients at the agriculture site input location.
